(19) United States
(12) Patent Application Publication
(10) Pub. No.: US 2007/0248597 A1
(43) Pub. Date: Oct. 25, 2007

(54) Methods of decreasing vascular calcification using IL-1 inhibitors

(76) Inventors: Charles M. Henley III, Thousand Oaks, CA (US); David Martin, Camarillo, CA (US)

Correspondence Address: AMGEN INC., MAIL STOP 28-2-C, ONE AMGEN CENTER DRIVE, THOUSAND OAKS, CA, 91320-1799, US

(21) Appl. No.: 11/584,034

(22) Filed: Oct. 20, 2006

Related U.S. Application Data
(60) Provisional application No. 60/729,305, filed on Oct. 21, 2005.

(51) Int. Cl. A61K 39/395; A61P 5/48; A61P 9/14

(52) U.S. Cl. 424/133.1

(57) ABSTRACT

The present invention relates to methods of decreasing, inhibiting, or preventing vascular calcification in subjects by administering IL-1 inhibitors. IL-1 inhibitors useful in this invention include anakinra, IL-1 antibodies, IL-1 RI antibodies, IL-1 trap molecules, soluble IL-1 receptors, and anti-IL-1/IL-1R peptides and peptibodies.

This application claims the benefit of U.S. Provisional Application No. 60/729,305, filed Oct. 21, 2005, which is hereby incorporated by reference.

### FIELD OF THE INVENTION

This invention relates generally to the field of medicine and, more specifically, to methods of decreasing, treating or preventing vascular calcification.

### BACKGROUND OF THE INVENTION

The IL-1 System

One of the most potent inflammatory cytokines yet discovered is interleukin-1 (IL-1). IL-1 is thought to be a key mediator in many diseases and medical conditions. It is manufactured (though not exclusively) by cells of the macrophage/monocyte lineage and may be produced in two forms: IL-1 alpha (IL-1α) and IL-1 beta (IL-1β). A third cytokine in the system acts as an antagonist and is referred to as IL-1 receptor antagonist (IL-1ra).

There are three known IL-1 receptor subunits. The active receptor complex consists of the type I receptor (IL-1 RI) and IL-1 receptor accessory protein (IL-1RAcP). IL-1 RI is responsible for binding the three naturally occurring ligands (IL-1 alpha, IL-1 beta and IL-1 ra) and is able to do so in the absence of the IL-1 RAcP. However, signal transduction requires interaction of IL-1 alpha or IL-1 beta with IL-1 RAcP. IL-1ra does not interact with the IL-1 RAcP and hence cannot signal. A third receptor subunit, the type II receptor (IL-1 RII), binds IL-1 alpha or IL-1 beta but cannot signal because it lacks an intracellular domain. Instead, it inhibits IL-1 bioactivity by acting as a decoy receptor in both a membrane-bound form and a cleaved, secreted form. See Dinarello (1996) Blood 87:2095-2147.

IL-1ra inhibits IL-1 alpha and IL-1 beta by binding to IL-1 RI but not transducing an intracellular signal or a biological response. IL-1ra inhibits the biological activities of IL-1 both in vitro and in vivo, and has been shown to be effective in animal models of septic shock, rheumatoid arthritis, graft versus host disease, stroke, and cardiac ischemia. A recombinant form of IL-1ra produced in E. coli is currently approved for pharmaceutical use in the United States and Europe. This drug has the generic name anakinra and is marketed under the trade name Kineret®.

Vascular Calcification

Vascular calcification, a well-recognized and common complication of chronic kidney disease (CKD), increases the risk of cardiovascular morbidity and mortality (Giachelli, C. J. Am. Soc. Nephrol. 15: 2959-64, 2004; Raggi, P. et al. J. Am. Coll. Cardiol. 39: 695-701, 2002). While the causes of vascular calcification in CKD remain to be elucidated, associated risk factors include age, gender, hypertension, time on dialysis, diabetes and glucose intolerance, obesity, and cigarette smoking (Zoccali C. Nephrol. Dial. Transplant 15: 454-7, 2000). These conventional risk factors, however, do not adequately explain the high mortality rates from cardiovascular causes in the patient population. Recent observations suggest that certain abnormalities in calcium and phosphorus metabolism, resulting in a raised serum calcium-phosphorus product (Ca×P) contribute to the development of arterial calcification, and possibly to cardiovascular disease, in patients with end-stage renal disease (Goodman, W. et al. N. Engl. J. Med. 342: 1478-83, 2000; Guerin, A. et al. Nephrol. Dial. Transplant 15:1014-21, 2000; Vattikuti, R. & Towler, D. Am. J. Physiol. Endocrinol. Metab. 286: E686-96, 2004).

Another hallmark of advanced CKD is secondary hyperparathyroidism (HPT), characterized by elevated parathyroid hormone (PTH) levels and disordered mineral metabolism. The elevations in calcium, phosphorus, and Ca×P observed in patients with secondary HPT have been associated with an increased risk of vascular calcification (Chertow, G. et al. Kidney Int. 62: 245-52, 2002; Goodman, W. et al. N. Engl. J. Med. 342: 1478-83, 2000; Raggi, P. et al. J. Am. Coll. Cardiol. 39: 695-701, 2002). Commonly used therapeutic interventions for secondary HPT, such as calcium-based phosphate binders and doses of active vitamin D sterols can result in hypercalcemia and hyperphosphatemia (Chertow, G. et al. Kidney Int. 62: 245-52, 2002; Tan, A. et al. Kidney Int 51: 317-23, 1997; Gallieni, M. et al. Kidney Int 42: 1191-8, 1992), which are associated with the development or exacerbation of vascular calcification.

Vascular calcification is an important and potentially serious complication of chronic renal failure. Two distinct patterns of vascular calcification have been identified (Proudfoot, D & Shanahan, C. Herz 26: 245-51, 2001), and it is common for both types to be present in uremic patients (Chen, N. & Moe, S. Semin Nephrol 24: 61-8, 2004). The first, medial calcification, occurs in the media of the vessel in conjunction with a phenotypic transformation of smooth muscle cells into osteoblast-like cells, while the other, atherogenesis, is associated with lipid-laden macrophages and intimal hyperplasia.

Medial wall calcification can develop in relatively young persons with chronic renal failure, and it is common in patients with diabetes mellitus even in the absence of renal disease. The presence of calcium in the medial wall of arteries distinguishes this type of vascular calcification from that associated with atherosclerosis (Schinke T. & Karsenty G. Nephrol Dial Transplant 15: 1272-4, 2000). Atherosclerotic vascular calcification occurs in atheromatous plaques along the intimal layer of arteries (Farzaneh-Far A. JAMA 284: 1515-6, 2000). Calcification is usually greatest in large, well-developed lesions, and it increases with age (Wexler L. et al. Circulation 94: 1175-92, 1996; Rumberger J. et al. Mayo Clin Proc 1999; 74: 243-52.). The extent of arterial calcification in patients with atherosclerosis generally corresponds to severity of disease. Unlike medial wall calcification, atherosclerotic vascular lesions, whether or not they contain calcium, impinge upon the arterial lumen and compromise blood flow. The localized deposition of calcium within atherosclerotic plaques may happen because of inflammation due to oxidized lipids and other oxidative stresses and infiltration by monocytes and macrophages (Berliner J. et al. Circulation 91: 2488-96, 1995).

Some patients with end-stage renal disease develop a severe form of occlusive arterial disease called calciphylaxis or calcific uremic arteriolopathy. This syndrome is characterized by extensive calcium deposition in small arteries (Gipstein R. et al. Arch Intern Med 136: 1273-80, 1976; Richens G. et al. J Am Acad. Dermatol. 6: 537-9, 1982). In patients with this disease, arterial calcification and vascular occlusion lead to tissue ischemia and necrosis. Involvement of peripheral vessels can cause ulceration of the skin of the lower legs or gangrene of the digits of the feet or hands. Ischemia and necrosis of the skin and subcutaneous adipose tissue of the abdominal wall, thighs and/or buttocks are features of a proximal form of calcific uremic arteriolopathy (Budisavljevic M. et al. J Am Soc Nephrol. 7: 978-82, 1996; Ruggian J. et al. Am. J. Kidney Dis. 28: 409-14, 1996). This syndrome occurs more frequently in obese individuals, and women are affected more often than men for reasons that remain unclear (Goodman W. J. Nephrol. 15(6): S82-S85, 2002).

Current therapies to normalize serum mineral levels or to decrease, inhibit, or prevent calcification of vascular tissues or implants are of limited efficacy and cause unacceptable side effects. Therefore, there exists a need for an effective method of inhibiting and preventing vascular calcification.

### SUMMARY OF THE INVENTION

The present invention provides methods of inhibiting, decreasing, or preventing vascular calcification in a subject comprising administering a therapeutically effective amount of an IL-1 inhibitor to the subject. In one aspect, the vascular calcification can be atherosclerotic calcification. In another aspect, the vascular calcification can be medial calcification.

In one aspect, the subject can be suffering from chronic renal insufficiency or end-stage renal disease. In another aspect, the subject can be pre-dialysis. In a further aspect, the subject can be suffering from uremia. In another aspect, the subject can be suffering from diabetes mellitus I or II. In another subject, the subject can be suffering from a cardiovascular disorder. In one aspect, the subject can be human.

In one aspect, the IL-1 inhibitor can be the molecule having the generic name anakinra. In another aspect, the IL-1 inhibitor can be a molecule having the sequence shown in FIG. 4 hereinafter (SEQ ID NO: 1), hereinafter referred to as “Fc-IL-1ra.” In yet another aspect, the IL-1 inhibitor can be an antibody to the IL-1 receptor. Preferred antibodies to the IL-1 receptor are described in U.S. Pat. App. 2004/0097712, published May 20, 2004 (U.S. Ser. No. 10/656,769). In a still further aspect, the IL-1 inhibitor can be an IL-1 trap molecule.

In one aspect, the IL-1 inhibitor used in the methods of the invention can be N-((6-(methyloxy)-4′-(trifluoromethyl)-1,1′-biphenyl-3-yl) methyl)-1-phenylethanamine, or a pharmaceutically acceptable salt thereof.

In one aspect, the invention provides methods of inhibiting, decreasing, or preventing vascular calcification, wherein a vitamin D sterol had been previously administered to the subject. In one aspect, the vitamin D sterol can be calcitriol, alfacalcidol, doxercalciferol, maxacalcitol or paricalcitol. In one aspect, the IL-1 inhibitor can be administered prior to or following administration of a vitamin D sterol. In another aspect, the IL-1 inhibitor can be administered in combination with a vitamin D sterol.

In one aspect, the IL-1 inhibitor can be administered in combination with RENAGEL®.

The invention further provides methods of decreasing serum creatinine levels in a subject, comprising administering a therapeutically effective of an IL-1 inhibitor to the subject. In one aspect, the subject can be suffering from increased serum creatinine levels induced by the administration of a vitamin D sterol to the subject.

### BRIEF DESCRIPTION OF THE FIGURES

FIG. 1 shows an experimental paradigm for adenine model of chronic kidney disease (CKD) and secondary hyperparathyroidism (SHPT).

FIG. 2 shows prevention of aortic vascular calcification with FC-IL-1ra in an animal model of CKD. The asterisk in FIG. 2 indicates that under the conditions tested (p=0.005; unpaired t-test; control (ASS) vehicle (red; n=4); Fc-IL-1ra (blue hatched, n=7)), there was no calcification (0 g/cm2 bone mineral density) in seven of seven Fc-IL-1 ra-treated animals.

FIG. 3 shows reduction of parathyroid gland size (A) and serum PTH (B) by Fc-IL-1ra in an animal model of CKD/SHPT. In FIG. 3A, the asterisk (*) denotes the parathyroid wild type/body wild type for A5S vehicle control vs. Fc-IL-1ra; p=0.009; unpaired t-test; control (vehicle, ASS), red; n=8); Fc-IL-1ra, (blue, n=13). In FIG. 3B, the pound sign (#) denotes serum PTH for A5S vehicle control vs. Fc-IL-1ra; p=0.028; unpaired t-test; control (vehicle, A5S), red; n=4); Fc-IL-1ra, (blue, n=7).

FIG. 4 shows the amino acid sequence (SEQ ID NO: 1) of the molecule referred to herein as Fc-IL-1ra.

FIG. 5 shows the experimental paradigm for the 5/6-nephrectomy model of CKD and secondary HPT.

FIG. 6 shows attenuation of calcitriol-induced aortic vascular calcification in uremic rats treated with Fc-IL-1ra.

### DETAILED DESCRIPTION OF THE INVENTION

Recent scientific literature includes suggestions to study the effects of cytokines on vascular calcification. Yao et al. (2004), Scandinavian J. Urol. Nephrol. 38: 405-16, found what they considered a “strong association between inflammation and increased oxidative stress and endothelial dysfunction” in end-stage renal disease (ESRD) patients. Malberti and Ravini (2005), Giornale Italiano di Nefrologia (22 Suppl.) 31: S47-52, noted that vascular calcifications are more frequent in dialysis patients than in the general population or in patients with cardiovascular disease with normal renal function, which led these authors to suggest study of the effects of anti-inflammatory treatments on the nutritional and cardiovascular status of ESRD patients. Moe and Chen (2005), Blood Purif. 23: 64-71, noted that cytokines and other mediators of inflammation may have a direct stimulatory effect on vascular calcification, leading them to suggest that inhibition of cytokine-mediated inflammation represents “a plausible therapeutic approach to limit vascular calcification.” The literature does not identify, however, which inflammatory cytokines may mediate vascular calcification.

Elsewhere in the literature, Nicklin et al. (2000), J. Exper. Med. 191: 303-11, found that IL-1ra deficient mice develop lethal arterial inflammation in flexpoints and branch points of the aorta. Arai et al. (1998), J. Toxicological Sciences 23: 121-8, found that 1,25 dihydroxyvitamin D₃ has been shown to increase IL-1 synthesis. The literature does not clarify, however, a definitive role for IL-1 receptor antagonism in prevention of vascular calcification.

The present invention is directed to methods of reducing, inhibiting, or preventing vascular calcification using IL-1 inhibitors.

IL-1 Inhibitors in General

“IL-1” refers to IL-1α and IL-1β.

“IL-1 inhibitors” as used throughout this specification refers to molecules that decrease the bioactivity of IL-1α, IL-1β, or IL-1 receptor type I (IL-1 RI), whether by direct or indirect interaction with IL-1α, IL-1β, IL-1 RI, IL-1 receptor accessory protein (IL-1RacP), interleukin-1 converting enzyme (ICE), with proteins that mediate signaling through a receptor for IL-1α or β, with proteins controlling the expression or release of IL-1α, IL-1β, IL-1 RI or IL-1 RII. Inhibition of IL-1 may result from a number of mechanisms, including down-regulation of IL-1 transcription, expression, or release from cells that produce IL-1; binding of free IL-1; interference with binding of IL-1 to its receptor; interference with formation of the IL-1 receptor complex (i.e., association of the IL-1 receptor type I with IL-1 RacP); and interference with modulation of IL-1 signaling after binding to its receptor. Thus, the term “IL-1 inhibitor” includes, but is not limited to, IL-1 beta inhibitors and IL-1 receptor antagonists (IL-1ra), such as anakinra and antibodies to IL-1 RI.

Classes of IL-1 inhibitors include the following, which are described in detail further hereinbelow:

Interleukin-1 receptor antagonists such as IL-1ra and anti-IL-1 receptor monoclonal antibodies, as described below;

IL-1 binding proteins such as soluble IL-1 receptors, anti-IL-1 monoclonal antibodies;

Inhibitors of interleukin-1 beta converting enzyme (ICE) or caspase I (e.g., WO 99/46248, WO 99/47545, and WO 99/47154, the disclosures of which are hereby incorporated by reference), which can be used to inhibit IL-1 beta production and secretion;

Interleukin-1 beta protease inhibitors;

and compounds and proteins that block in vivo synthesis or extracellular release of IL-1.

The term “IL-1 binding proteins” refers to molecules that bind to IL-1 and thus prevent IL-1 beta from exerting bioactivity when bound to IL-1 RI. Thus, IL-1 beta inhibitors include, but are not limited to, IL-1 beta antibodies, peptides that bind to IL-1 beta, peptibodies that bind to IL-1 beta, soluble IL-1 receptor molecules, and IL-1 trap molecules.

The term “IL-1 receptor antagonists” refers to molecules that bind to IL-1 R1 or IL-1 RacP or otherwise prevent the interaction of IL-1 RI and IL-1 RacP. Thus, the term “IL-1 receptor antagonists” includes, but is not limited to anakinra, Fc-IL-1ra, IL-1 RI antibodies, IL-1RacP antibodies, peptides that bind to IL-1 RI or to IL-1 RAcP, and peptibodies that bind to IL-1 RI or IL-1 RAcP.

Exemplary IL-1 inhibitors are disclosed in the following references:

U.S. Pat. Nos. 5,747,444; 5,359,032; 5,608,035; 5,843,905; 5,359,032; 5,866,576; 5,869,660; 5,869,315; 5,872,095; 5,955,480; 5,965,564;

International (WO) patent applications 98/21957, 96/09323, 91/17184, 96/40907, 98/32733, 98/42325, 98/44940, 98/47892, 98/56377, 99/03837, 99/06426, 99/06042, 91/17249, 98/32733, 98/17661, 97/08174, 95/34326, 99/36426, 99/36415;

European (EP) patent applications 534978 and 894795; and

French patent application FR 2762514.

IL-1 Receptor Antagonists

For purposes of the present invention, IL-1ra and variants and derivatives thereof as discussed hereinafter are collectively termed “IL-1ra protein(s)”. The molecules described in the above references and the variants and derivatives thereof discussed hereinafter are collectively termed “IL-1 inhibitors.”

IL-1ra is a human protein that acts as a natural inhibitor of interleukin-1 and which is a member of the IL-1 family member that includes IL-1α and IL-1β. Preferred receptor antagonists (including IL-1ra and variants and derivatives thereof), as well as methods of making and using thereof, are described in WO 91/08285; WO 91/17184; AU 9173636; WO 92/16221; WO93/21946; WO 94/06457; WO 94/21275; FR 2706772; WO 94/21235; DE 4219626, WO 94/20517; WO 96/22793; WO 97/28828; WO 99/36541, and U.S. Pat. Nos. 5,075,222 and 6,599,873 (incorporated herein by reference). The proteins include glycosylated as well as non-glycosylated IL-1 receptor antagonists.

Specifically, three useful forms of IL-1ra and variants thereof are disclosed and described in the U.S. Pat. No. 5,075,222 patent. The first of these, called “IL-1i” in the '222 patent, is characterized as a 22-23 kD molecule on SDS-PAGE with an approximate isoelectric point of 4.8, eluting from a Mono Q FPLC column at around 52 mM NaCl in Tris buffer, pH 7.6. The second, IL-1raβ, is characterized as a 22-23 kD protein, eluting from a Mono Q column at 48 mM NaCl. Both IL-1raα and IL-1raβ are glycosylated. The third, IL-1rax, is characterized as a 20 kD protein, eluting from a Mono Q column at 48 mM NaCl, and is non-glycosylated. U.S. Pat. No. 5,075,222 patent also discloses methods for isolating the genes responsible for coding the inhibitors, cloning the gene in suitable vectors and cell types, and expressing the gene to produce the inhibitors.

Those skilled in the art understand that many combinations of deletions, insertions and substitutions (individually or collectively “variant(s)”) can be made within the amino acid sequences of IL-1ra, provided that the resulting molecule is biologically active (e.g., possesses the ability to inhibit IL-1). Particular variants are described in U.S. Pat. No. 5,075,222 and U.S. Ser. No. 11/097,453, which are hereby incorporated by reference.

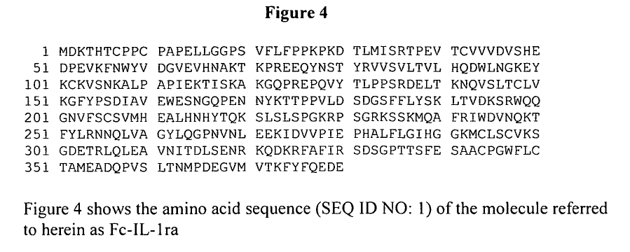

The term “IL-1 receptor antagonist” further includes modified IL-1ra and fusion proteins comprising IL-1ra. Exemplary fusion proteins include Fc-IL-1ra (FIG. 4, SEQ ID NO: 1), and molecules as described in U.S. Pat. No. 6,294,170.

Antibodies

“IL-1 beta antibodies” and “antibodies to IL-1 beta” refer to antibodies that specifically bind to IL-1 beta. One example of an IL-1 beta antibody is known as MAb 201 and is commercially available. Additional IL-1 beta antibodies may be produced as described hereinafter. Further examples of IL-1 antibodies are described in WO 9501997, WO 9402627, WO 9006371, EP 364778, EP 267611, EP 220063, and U.S. Pat. No. 4,935,343 (incorporated by reference).

Antibodies having specific binding affinity for IL-1β can be produced through standard methods. Alternatively, antibodies may be commercially available, for example, from R&D Systems, Inc., Minneapolis, Minn. The terms “antibody” and “antibodies” include polyclonal antibodies, monoclonal antibodies, humanized or chimeric antibodies, single chain Fv antibody fragments, Fab fragments, and F(ab)₂ fragments. Polyclonal antibodies are heterogeneous populations of antibody molecules that are specific for a particular antigen, which are contained in the sera of the immunized animals. Polyclonal antibodies are produced using well-known methods.

Likewise, “IL-1 RI antibodies” and “antibodies to IL-1 RI” refer to antibodies that specifically bind to IL-1 RI. Examples of IL-1 RII antibodies are described in EP 623 674 and U.S. Pat. App. 2004/0097712, published May 20, 2004 (U.S. Ser. No. 10/656,769), the disclosure of which is hereby incorporated by reference. Additional IL-1 RI antibodies may be produced as described hereinafter.

The terms “antibody” and “antibodies” as used herein refer to intact antibody, or a binding fragment thereof that competes with the intact antibody for specific binding and includes chimeric, humanized, fully human, and bispecific antibodies. In certain embodiments, binding fragments are produced by recombinant DNA techniques. In additional embodiments, binding fragments are produced by enzymatic or chemical cleavage of intact antibodies. Binding fragments include, but are not limited to, Fab, Fab′, F(ab′)₂, Fv, and single-chain antibodies.

The term “heavy chain” includes a full-length heavy chain and fragments thereof having sufficient variable region sequence to confer specificity for IL-1RI. A full-length heavy chain includes a variable region domain, V_{H}, and three constant region domains, C_{H}1, C_{H}2, and C_{H}3. The V_{H} domain is at the amino-terminus of the polypeptide, and the C_{H}3 domain is at the carboxyl-terminus.

The term “light chain” includes a full-length light chain and fragments thereof having sufficient variable region sequence to confer specificity for IL-1 RI. A full-length light chain includes a variable region domain, V_{L}, and a constant region domain, C_{L}. Like the heavy chain, the variable region domain of the light chain is at the amino-terminus of the polypeptide.

Monoclonal antibodies, which are homogeneous populations of antibodies to a particular epitope contained within an antigen, can be prepared using standard hybridoma technology. In particular, monoclonal antibodies can be obtained by any technique that provides for the production of antibody molecules by continuous cell lines in culture such as described by Kohler, G. et al., Nature, 1975, 256:495, the human B-cell hybridoma technique (Kosbor et al., Immunology Today, 1983, 4:72; Cole et al., Proc. Natl. Acad. Sci. USA, 1983, 80:2026), and the EBV-hybridoma technique (Cole et al., Monoclonal Antibodies and Cancer Therapy, Alan R. Liss, Inc., 1983, pp. 77-96). Such antibodies can be of any immunoglobulin class including IgG, IgM, IgE, IgA, IgD, and any subclass thereof. The hybridoma producing the monoclonal antibodies of the invention can be cultivated in vitro or in vivo.

A chimeric antibody is a molecule in which different portions are derived from different animal species, such as those having a variable region derived from a murine monoclonal antibody and a human immunoglobulin constant region. Chimeric antibodies can be produced through standard techniques.

Antibody fragments that have specific binding affinity for IL-1β can be generated by known techniques. For example, such fragments include, but are not limited to, F(ab′)₂ fragments that can be produced by pepsin digestion of the antibody molecule, and Fab fragments that can be generated by reducing the disulfide bridges of F(ab′)₂ fragments. Alternatively, Fab expression libraries can be constructed. See, for example, Huse et al., 1989, Science, 246: 1275. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques. See, for example, U.S. Pat. No. 4,946,778.

A “Fab fragment” is comprised of one light chain and the CHI and variable regions of one heavy chain. The heavy chain of a Fab molecule cannot form a disulfide bond with another heavy chain molecule.

A “Fab′ fragment” contains one light chain and one heavy chain that contains more of the constant region, between the C_{H}1 and C_{H}2 domains, such that an interchain disulfide bond can be formed between two heavy chains to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between two heavy chains.

The “Fv region” comprises the variable regions from both the heavy and light chains, but lacks the constant regions.

“Single-chain antibodies” are Fv molecules in which the heavy and light chain variable regions have been connected by a flexible linker to form a single polypeptide chain, which forms an antigen-binding region. Single chain antibodies are discussed in detail in International Patent Application Publication No. WO 88/01649 and U.S. Pat. Nos. 4,946,778 and 5,260,203 (hereby incorporated by reference).

A “bivalent antibody” other than a “multispecific” or “multifunctional” antibody, in certain embodiments, is understood to comprise binding sites having identical antigenic specificity.

A “bispecific” or “bifunctional” antibody is a hybrid antibody having two different heavy/light chain pairs and two different binding sites. Bispecific antibodies may be produced by a variety of methods including, but not limited to, fusion of hybridomas or linking of Fab′ fragments. See, e.g., Songsivilai & Lachmann (1990), Clin. Exp. Immunol. 79:315-321; Kostelny et al. (1992), J. Immunol. 148:1547-1553.

Preferred antibodies are described in U.S. Pat. App. 2004/0097712, published May 20, 2004 (hereinafter referred to as the '712 application). Specifically preferred are antibodies having a heavy chain variable region selected from the following:

(SEQ ID NO: 408)

Met Glu Phe Gly Leu Ser Trp Val Phe Leu 10
Val Ala Leu Leu Arg Gly Val Gln Cys Gln 20
Val Gln Leu Val Glu Ser Gly Gly Gly Val 30
Val Gln Pro Gly Arg Ser Leu Arg Leu Ser 40
Cys Ala Ala Ser Gly Phe Thr Phe Ser Asn 50
Tyr Gly Met His Trp Val Arg Gln Ala Pro 60
Gly Lys Gly Leu Glu Trp Val Ala Gly Ile 70
Trp Asn Asp Gly Ile Asn Lys Tyr His Ala 80
His Ser Val Arg Gly Arg Phe Thr Ile Ser 90
Arg Asp Asn Ser Lys Asn Thr Leu Tyr Leu 100
Gln Met Asn Ser Pro Arg Ala Glu Asp Thr 110
Ala Val Tyr Tyr Cys Ala Arg Ala Arg Ser 120
Phe Asp Trp Leu Leu Phe Glu Phe Trp Gly 130
Gln Gly Thr Leu Val Thr Val Ser Ser 139

(SEQ ID NO: 409)

Met Glu Phe Gly Leu Ser Trp Val Phe Leu 10
Val Ala Leu Leu Arg Gly Val Gln Cys Gln 20
Val Gln Leu Val Glu Ser Gly Gly Gly Val 30
Val Gln Pro Gly Arg Ser Leu Arg Leu Ser 40
Cys Ala Val Ser Gly Phe Thr Phe Ser Asn 50
Tyr Gly Met His Trp Val Arg Gln Ala Pro 60
Gly Lys Gly Leu Glu Trp Val Ala Ala Ile 70
Trp Asn Asp Gly Glu Asn Lys His His Ala 80
Gly Ser Val Arg Gly Arg Phe Thr Ile Ser 90
Arg Asp Asn Ser Lys Asn Thr Leu Tyr Leu 100
Gln Met Asn Ser Leu Arg Ala Glu Asp Thr 110
Ala Val Tyr Tyr Cys Ala Arg Gly Arg Tyr 120
Phe Asp Trp Leu Leu Phe Glu Tyr Trp Gly 130
Gln Gly Thr Leu Val Thr Val Ser Ser 139

(SEQ ID NO: 410)

Met Gly Ser Thr Ala Ile Leu Ala Leu Leu 10
Leu Ala Val Leu Gln Gly Val Cys Ala Glu 20
Val Gln Leu Met Gln Ser Gly Ala Glu Val 30
Lys Lys Pro Gly Glu Ser Leu Lys Ile Ser 40
Cys Lys Gly Ser Gly Tyr Ser Phe Ser Phe 50
His Trp Ile Ala Trp Val Arg Gln Met Pro 60
Gly Lys Gly Leu Glu Trp Met Gly Ile Ile 70
His Pro Gly Ala Ser Asp Thr Arg Tyr Ser 80
Pro Ser Phe Gln Gly Gln Val Thr Ile Ser 90
Ala Asp Asn Ser Asn Ser Ala Thr Tyr Leu 100
Gln Trp Ser Ser Leu Lys Ala Ser Asp Thr 110
Ala Met Tyr Phe Cys Ala Arg Gln Arg Glu 120
Leu Asp Tyr Phe Asp Tyr Trp Gly Gln Gly 130
Thr Leu Val Thr Val Ser Ser 137

The foregoing are SEQ ID NOS: 10, 14, and 16 of the '712 application. Antibodies incorporating these sequences may be prepared as described therein. Most preferred are antibodies having all or an immunologically functional fragment of a heavy chain having a sequence selected from SEQ ID NOS: 20, 22, 24, 26, 28, 30, 32, 34, and 36 of the '712 application, all of which are specifically incorporated by reference.

Also specifically preferred are antibodies having a light chain variable region selected from the following:

(SEQ ID NO: 411)

Met Glu Ala Pro Ala Gln Leu Leu Phe Leu 10
Leu Leu Leu Trp Leu Pro Asp Thr Thr Gly 20
Glu Ile Val Leu Thr Gln Ser Pro Ala Thr 30
Leu Ser Leu Ser Pro Gly Glu Arg Ala Thr 40
Leu Ser Cys Arg Ala Ser Gln Ser Val Ser 50
Ser Tyr Leu Ala Trp Tyr Gln Gln Lys Pro 60
Gly Gln Ala Pro Arg Leu Leu Ile Tyr Asp 70
Ala Ser Asn Arg Ala Thr Gly Ile Pro Ala 80
Arg Phe Ser Gly Ser Gly Ser Gly Thr Asp 90
Phe Thr Leu Thr Ile Ser Ser Leu Glu Pro 100
Glu Asp Phe Ala Val Tyr Tyr Cys Gln Gln 110
Arg Ser Asn Trp Pro Pro Leu Thr Phe Gly 120
Gly Gly Thr Lys Val Glu Ile Lys 128

(SEQ ID NO: 412)

Met Ser Pro Ser Gln Leu Ile Gly Phe Leu 10
Leu Leu Trp Val Pro Ala Ser Arg Gly Glu 20
Ile Val Leu Thr Gln Ser Pro Asp Phe Gln 30
Ser Val Thr Pro Lys Glu Lys Val Thr Ile 40
Thr Cys Arg Ala Ser Gln Ser Ile Gly Ser 50
Ser Leu His Trp Tyr Gln Gln Lys Pro Asp 60
Gln Ser Pro Lys Leu Leu Ile Lys Tyr Ala 70
Ser Gln Ser Phe Ser Gly Val Pro Ser Arg 80
Phe Ser Gly Ser Gly Ser Gly Thr Asp Phe 90
Thr Leu Thr Ile Asn Ser Leu Glu Ala Glu 100
Asp Ala Ala Ala Tyr Tyr Cys His Gln Ser 110
Ser Ser Leu Pro Leu Thr Phe Gly Gly Gly 120
Thr Lys Val Glu Ile Lys 126

The foregoing are SEQ ID NOS: 12 and 18 of the '712 application. Antibodies incorporating these sequences may be prepared as described therein. Most preferred are antibodies having all or an immunologically functional fragment of a light chain having a sequence selected from SEQ ID NOS: 38 and 40 of the '712 application, all of which are specifically incorporated by reference.

Although SEQ ID NOS: 408 through 410 are described as heavy chain variable regions, persons skilled in the art may employ those sequences for IL-1R binding at a different position in an antibody (e.g., as a light chain variable region) or in another molecular (e.g., an Fc fusion molecule). Likewise, although SEQ ID NOS: 411 and 4112 are described as light chain variable regions, persons skilled in the art may employ those sequences for IL-1R binding at a different position in an antibody (e.g., as a heavy chain variable region) or in another molecular (e.g., an Fc fusion molecule). The same techniques can be used to prepare additional IL-1 beta antibodies or molecules derived from IL-1 beta antibodies. Thus, the MAb201 heavy and light chain variable regions can be used at different positions in an antibody framework and in different molecular forms. All such molecules described in this paragraph are within the scope of this invention.

Peptides and Peptibodies

Phage display peptide libraries have emerged as a powerful method in identifying peptide agonists and antagonists of proteins of interest. See, for example, Scott et al. (1990), Science 249: 386; Devlin et al. (1990), Science 249: 404; WO 96/40987, published Dec. 19, 1996; WO 98/15833, published Apr. 16, 1998; and U.S. Pat. Nos. 5,223,409; 5,733,731; 5,498,530; 5,432,018; 5,338,665; and 5,922,545 (each of which is incorporated by reference). In such libraries, random peptide sequences are displayed by fusion with coat proteins of filamentous phage. Typically, the displayed peptides are affinity-eluted against an antibody-immobilized extracellular domain of a receptor. The retained phages may be enriched by successive rounds of affinity purification and repropagation. The best binding peptides may be sequenced to identify key residues within one or more structurally related families of peptides. See, e.g., Cwirla et al. (1997), Science 276: 1696-9, in which two distinct families were identified. The peptide sequences may also suggest that residues may be safely replaced by alanine scanning or by mutagenesis at the DNA level. Mutagenesis libraries may be created and screened to further optimize the sequence of the best binders. Lowman (1997), Ann. Rev. Biophys. Biomol. Struct. 26: 401-24.

Phage display and other techniques may be used to generate peptide IL-1 inhibitors. Such peptides have been generated as described in U.S. Pat. Nos. 5,608,035, 5,786,331, 5,880,096, and 6,660,843, each of which is hereby incorporated by reference. Such peptides may be linked to Fc domains, polyethylene glycol, or other half-life extending moieties (see U.S. Pat. No. 6,660,843). Such peptides linked to Fc domains are referred to as “peptibodies.” Peptibodies directed to targets other than IL-1 and IL-1 receptor have shown efficacy in human clinical trials. A number of peptides suitable for use in peptibodies are described in Table 1 below.

TABLE 1
IL-1 antagonist peptide sequences

| | |
|---|---|
| | SEQ |
| | ID |
| Sequence/structure | NO: |

| | |
|---|---|
| TANVSSFEWTPYYWQPYALPL | 2 |
| SWTDYGYWQPYALPISGL | 3 |
| ETPFTWEESNAYYWQPYALPL | 4 |
| ENTYSPNWADSMYWQPYALPL | 5 |
| SVGEDHNFWTSEYWQPYALPL | 6 |
| DGYDRWRQSGERYWQPYALPL | 7 |
| FEWTPGYWQPY | 8 |
| FEWTPGYWQHY | 9 |
| FEWTPGWYQJY | 10 |
| AcFEWTPGWYQJY | 11 |
| FEWTPGWpYQJY | 12 |
| FAWTPGYWQJY | 13 |
| FEWAPGYWQJY | 14 |
| FEWVPGYWQJY | 15 |
| FEWTPGYWQJY | 16 |
| AcFEWTPGYWQJY | 17 |
| FEWTPaWYQJY | 18 |
| FEWTPSarWYQJY | 19 |
| FEWTPGYYQPY | 20 |
| FEWTPGWWQPY | 21 |
| FEWTPNYWQPY | 22 |
| FEWTPvYWQJY | 23 |
| FEWTPecGYWQJY | 24 |
| FEWTPAibYWQJY | 25 |
| FEWTSarGYWQJY | 26 |
| FEWTPGYWQPY | 27 |
| FEWTPGYWQHY | 28 |
| FEWTPGWYQJY | 29 |
| AcFEWTPGWYQJY | 30 |
| FEWTPGW-pY-QJY | 31 |
| FAWTPGYWQJY | 32 |
| FEWAPGYWQJY | 33 |
| FEWVPGYWQJY | 34 |
| FEWTPGYWQJY | 35 |
| AcFEWTPGYWQJY | 36 |
| FEWTPAWYQJY | 37 |
| FEWTPSarWYQJY | 38 |
| FEWTPGYYQPY | 39 |
| FEWTPGWWQPY | 40 |
| FEWTPNYWQPY | 41 |
| FEWTPVYWQJY | 42 |
| FEWTPecGYWQJY | 43 |
| FEWTPAibYWQJY | 44 |
| FEWTSarGYWQJY | 45 |
| FEWTPGYWQPYALPL | 46 |
| 1NapEWTPGYYQJY | 47 |
| YEWTPGYYQJY | 48 |
| FEWVPGYYQJY | 49 |
| FEWTPSYYQJY | 50 |
| FEWTPNYYQJY | 51 |
| TKPR | 52 |
| RKSSK | 53 |
| RKQDK | 54 |
| NRKQDK | 55 |
| RKQDKR | 56 |
| ENRKQDKRF | 57 |
| VTKFYF | 58 |
| VTKFY | 59 |
| VTDFY | 60 |
| SHLYWQPYSVQ | 61 |
| TLVYWQPYSLQT | 62 |
| RGDYWQPYSVQS | 63 |
| VHVYWQPYSVQT | 64 |
| RLVYWQPYSVQT | 65 |
| SRVWFQPYSLQS | 66 |
| NMVYWQPYSIQT | 67 |
| SVVFWQPYSVQT | 68 |
| TFVYWQPYALPL | 69 |
| TLVYWQPYSIQR | 70 |
| RLVYWQPYSVQR | 71 |
| SPVFWQPYSIQI | 72 |
| WIEWWQPYSVQS | 73 |
| SLIYWQPYSLQM | 74 |
| TRLYWQPYSVQR | 75 |
| RCDYWQPYSVQT | 76 |
| MRVFWQPYSVQN | 77 |
| KIVYWQPYSVQT | 78 |
| RHLYWQPYSVQR | 79 |
| ALVWWQPYSEQI | 80 |
| SRVWFQPYSLQS | 81 |
| WEQPYALPLE | 82 |
| QLVWWQPYSVQR | 83 |
| DLRYWQPYSVQV | 84 |
| ELVWWQPYSLQL | 85 |
| DLVWWQPYSVQW | 86 |
| NGNYWQPYSFQV | 87 |
| ELVYWQPYSIQR | 88 |
| ELMYWQPYSVQE | 89 |
| NLLYWQPYSMQD | 90 |
| GYEWYQPYSVQR | 91 |
| SRVWYQPYSVQR | 92 |
| LSEQYQPYSVQR | 93 |
| GGGWWQPYSVQR | 94 |
| VGRWYQPYSVQR | 95 |
| VHVYWQPYSVQR | 96 |
| QARWYQPYSVQR | 97 |
| VHVYWQPYSVQT | 98 |
| RSVYWQPYSVQR | 99 |
| TRVWFQPYSVQR | 100 |
| GRIWFQPYSVQR | 101 |
| GRVWFQPYSVQR | 102 |
| ARTWYQPYSVQR | 103 |
| ARVWWQPYSVQM | 104 |
| RLMFYQPYSVQR | 105 |
| ESMWYQPYSVQR | 106 |
| HFGWWQPYSVHM | 107 |
| ARFWWQPYSVQR | 108 |
| RLVYWQ PYAPIY | 109 |
| RLVYWQ PYSYQT | 110 |
| RLVYWQ PYSLPI | 111 |
| RLVYWQ PYSVQA | 112 |
| SRVWYQ PYAKGL | 113 |
| SRVWYQ PYAQGL | 114 |
| SRVWYQ PYAMPL | 115 |
| SRVWYQ PYSVQA | 116 |
| SRVWYQ PYSLGL | 117 |
| SRVWYQ PYAREL | 118 |
| SRVWYQ PYSRQP | 119 |
| SRVWYQ PYFVQP | 120 |
| EYEWYQ PYALPL | 121 |
| IPEYWQ PYALPL | 122 |
| SRIWWQ PYALPL | 123 |
| DPLFWQ PYALPL | 124 |
| SRQWVQ PYALPL | 125 |
| IRSWWQ PYALPL | 126 |
| RGYWQ PYALPL | 127 |
| RLLWVQ PYALPL | 128 |
| EYRWFQ PYALPL | 129 |
| DAYWVQ PYALPL | 130 |
| WSGYFQ PYALPL | 131 |
| NIEFWQ PYALPL | 132 |
| TRDWVQ PYALPL | 133 |
| DSSWYQ PYALPL | 134 |
| IGNWYQ PYALPL | 135 |
| NLRWDQ PYALPL | 136 |
| LPEFWQ PYALPL | 137 |
| DSYWWQ PYALPL | 138 |
| RSQYYQ PYALPL | 139 |
| ARFWLQ PYALPL | 140 |
| NSYFWQ PYALPL | 141 |
| RFMYWQPYSVQR | 142 |
| AHLFWQPYSVQR | 143 |
| WWQPYALPL | 144 |
| YYQPYALPL | 145 |
| YFQPYALGL | 146 |
| YWYQPYALPL | 147 |
| RWWQPYATPL | 148 |
| GWYQPYALGF | 149 |
| YWYQPYALGL | 150 |
| IWYQPYAMPL | 151 |
| SNMQPYQRLS | 152 |
| TFVYWQPY AVGLPAAETACN | 153 |
| TFVYWQPY SVQMTITGKVTM | 154 |
| TFVYWQPY SSHXXVPXGFPL | 155 |
| TFVYWQPY YGNPQWAIHVRH | 156 |
| TFVYWQPY VLLELPEGAVRA | 157 |
| TFVYWQPY VDYVWPIPIAQV | 158 |
| GWYQPYVDGWR | 159 |
| RWEQPYVKDGWS | 160 |
| EWYQPYALGWAR | 161 |
| GWWQPYARGL | 162 |
| LFEQPYAKALGL | 163 |
| GWEQPYARGLAG | 164 |
| AWVQPYATPLDE | 165 |
| MWYQPYSSQPAE | 166 |
| GWTQPYSQQGEV | 167 |
| DWFQPYSIQSDE | 168 |
| PWIQPYARGFG | 169 |
| RPLYWQPYSVQV | 170 |
| TLIYWQPYSVQI | 171 |
| RFDYWQPYSDQT | 172 |
| WHQFVQPYALPL | 173 |
| EWDS VYWQPYSVQ TLLR | 174 |
| WEQN VYWQPYSVQ SFAD | 175 |
| SDV VYWQPYSVQ SLEM | 176 |
| YYDG VYWQPYSVQ VMPA | 177 |
| SDIWYQ PYALPL | 178 |
| QRIWWQ PYALPL | 179 |
| SRIWWQ PYALPL | 180 |
| RSLYWQ PYALPL | 181 |
| TIIWEQ PYALPL | 182 |
| WETWYQ PYALPL | 183 |
| SYDWEQ PYALPL | 184 |
| SRIWCQ PYALPL | 185 |
| EIMFWQ PYALPL | 186 |
| DYVWQQ PYALPL | 187 |
| MDLLVQ WYQPYALPL | 188 |
| GSKVIL WYQPYALPL | 189 |
| RQGANI WYQPYALPL | 190 |
| GGGDEP WYQPYALPL | 191 |
| SQLERT WYQPYALPL | 192 |
| ETWVRE WYQPYALPL | 193 |
| KKGSTQ WYQPYALPL | 194 |
| LQARMN WYQPYALPL | 195 |
| EPRSQK WYQPYALPL | 196 |
| VKQKWR WYQPYALPL | 197 |
| LRRHDV WYQPYALPL | 198 |
| RSTASI WYQPYALPL | 199 |
| ESKEDQ WYQPYALPL | 200 |
| EGLTMK WYQPYALPL | 201 |
| EGSREG WYQPYALPL | 202 |
| VIEWWQ PYALPL | 203 |
| VWYWEQ PYALPL | 204 |
| ASEWWQ PYALPL | 205 |
| FYEWWQ PYALPL | 206 |
| EGWWVQ PYALPL | 207 |
| WGEWLQ PYALPL | 208 |
| DYVWEQ PYALPL | 209 |
| AHTWWQ PYALPL | 210 |
| FIEWFQ PYALPL | 211 |
| WLAWEQ PYALPL | 212 |
| VMEWWQPYALPL | 213 |
| ERMWQPYALPL | 214 |
| NXXWXXPYALPL | 215 |
| WGNWYQPYALPL | 216 |
| TLYWEQPYALPL | 217 |
| VWRWEQPYALPL | 218 |
| LLWTQPYALPL | 219 |
| SRIWXXPYALPL | 220 |
| SDIWYQPYALPL | 221 |
| WGYYXXPYALPL | 222 |
| TSGWYQPYALPL | 223 |
| VHPYXXPYALPL | 224 |
| EHSYFQPYALPL | 225 |
| XXIWYQPYALPL | 226 |
| AQLHSQPYALPL | 227 |
| WANWFQPYALPL | 228 |
| SRLYSQ YALPL | 229 |
| GVTFSQPYALPL | 230 |
| SIVWSQPYALPL | 231 |
| SRDLVQPYALPL | 232 |
| HWGHVYWQPYSVQ DDLG | 233 |
| SWHSVYWQPYSVQ SVPE | 234 |
| WRDSVYWQPYSVQ PESA | 235 |
| TWDAVYWQPYSVQ KWLD | 236 |
| TPPWVYWQPYSVQ SLDP | 237 |
| YWSSVYWQPYSVQ SVHS | 238 |
| YWYQPYALGL | 239 |
| YWYQPY ALPL | 240 |
| EWIQPYATGL | 241 |
| NWEQPYAKPL | 242 |
| AFYQPYALPL | 243 |
| FLYQPYALPL | 244 |
| VCKQPYLEWC | 245 |
| ETPFTWEESNAYYWQPYALPL | 246 |
| QGWLTWQDSVDMYWQPYALPL | 247 |
| FSEAGYTWPENTYWQPYALPL | 248 |
| TESPGGLDWAKIYWQPYALPL | 249 |
| DGYDRWRQSGERYWQPYALPL | 250 |
| TANVSSFEWTPGYWQPYALPL | 251 |
| SVGEDHNFWTSEYWQPYALPL | 252 |
| MNDQTSEVSTFPYWQPYALPL | 253 |
| SWSEAFEQPRNLYWQPYALPL | 254 |
| QYAEPSALNDWGYWQPYALPL | 255 |
| NGDWATADWSNYYWQPYALPL | 256 |
| THDEHIYWQPYALPL | 257 |
| MLEKTYTTWTPGYWQPYALPL | 258 |
| WSDPLTRDADLYWQPYALPL | 259 |
| SDAFTTQDSQAMYWQPYALPL | 260 |
| GDDAAWRTDSLTYWQPYALPL | 261 |
| AIIRQLYRWSEMYWQPYALPL | 262 |
| ENTYSPNWADSMYWQPYALPL | 263 |
| MNDQTSEVSTFPYWQPYALPL | 264 |
| SVGEDHNFWTSEYWQPYALPL | 265 |
| QTPFTWEESNAYYWQPYALPL | 266 |
| ENPFTWQESNAYYWQPYALPL | 267 |
| VTPFTWEDSNVFYWQPYALPL | 268 |
| QIPFTWEQSNAYYWQPYALPL | 269 |
| QAPLTWQESAAYYWQPYALPL | 270 |
| EPTFTWEESKATYWQPYALPL | 271 |
| TTTLTWEESNAYYWQPYALPL | 272 |
| ESPLTWEESSALYWQPYALPL | 273 |
| ETPLTWEESNAYYWQPYALPL | 274 |
| EATFTWAESNAYYWQPYALPL | 275 |
| EALFTWKESTAYYWQPYALPL | 276 |
| STP-TWEESNAYYWQPYALPL | 277 |
| ETPFTWEESNAYYWQPYALPL | 278 |
| KAPFTWEESQAYYWQPYALPL | 279 |
| STSFTWEESNAYYWQPYALPL | 280 |
| DSTFTWEESNAYYWQPYALPL | 281 |
| YIPFTWEESNAYYWQPYALPL | 282 |
| QTAFTWEESNAYYWQPYALPL | 283 |
| ETLFTWEESNATYWQPYALPL | 284 |
| VSSFTWEESNAYYWQPYALPL | 285 |
| QPYALPL | 286 |
| Py-1-NapPYQJYALPL | 287 |
| TANVSSFEWTPG YWQPYALPL | 288 |
| FEWTPGYWQPYALPL | 289 |
| FEWTPGYWQJYALPL | 290 |
| FEWTPGYYQJYALPL | 291 |
| ETPFTWEESNAYYWQPYALPL | 292 |
| FTWEESNAYYWQJYALPL | 293 |
| ADVL YWQPYA PVTLWV | 294 |
| GDVAE YWQPYA LPLTSL | 295 |
| SWTDYG YWQPYA LPISGL | 296 |
| FEWTPGYWQPYALPL | 297 |
| FEWTPGYWQJYALPL | 298 |
| FEWTPGWYQPYALPL | 299 |
| FEWTPGWYQJYALPL | 300 |
| FEWTPGYYQPYALPL | 301 |
| FEWTPGYYQJYALPL | 302 |
| TANVSSFEWTPGYWQPYALPL | 303 |
| SWTDYGYWQPYALPISGL | 304 |
| ETPFTWEESNAYYWQPYALPL | 305 |
| ENTYSPNWADSMYWQPYALPL | 306 |
| SVGEDHNFWTSEYWQPYALPL | 307 |
| DGYDRWRQSGERYWQPYALPL | 308 |
| FEWTPGYWQPYALPL | 309 |
| FEWTPGYWQPY | 310 |
| FEWTPGYWQJY | 311 |
| EWTPGYWQPY | 312 |
| FEWTPGWYQJY | 313 |
| AEWTPGYWQJY | 314 |
| FAWTPGYWQJY | 315 |
| FEATPGYWQJY | 316 |
| FEWAPGYWQJY | 317 |
| FEWTAGYWQJY | 318 |
| FEWTPAYWQJY | 319 |
| FEWTPGAWQJY | 320 |
| FEWTPGYAQJY | 321 |
| FEWTPGYWQJA | 322 |
| FEWTGGYWQJY | 323 |
| FEWTPGYWQJY | 324 |
| FEWTJGYWQJY | 325 |
| FEWTPecGYWQJY | 326 |
| FEWTPAibYWQJY | 327 |
| FEWTPSarWYQJY | 328 |
| FEWTSarGYWQJY | 329 |
| FEWTPNYWQJY | 330 |
| FEWTPVYWQJY | 331 |
| FEWTVPYWQJY | 332 |
| AcFEWTPGWYQJY | 333 |
| AcFEWTPGYWQJY | 334 |
| INap-EWTPGYYQJY | 335 |
| YEWTPGYYQJY | 336 |
| FEWVPGYYQJY | 337 |
| FEWTPGYYQJY | 338 |
| FEWTPsYYQJY | 339 |
| FEWTPnYYQJY | 340 |
| SHLY-Nap-QPYSVQM | 341 |
| TLVY-Nap-QPYSLQT | 342 |
| RGDY-Nap-QPYSVQS | 343 |
| NMVY-Nap-QPYSIQT | 34A |
| VYWQPYSVQ | 345 |
| VY-Nap-QPYSVQ | 346 |
| TFVYWQJYALPL | 347 |
| FEWTPGYYQJ-Bpa | 348 |
| XaaFEWTPGYYQJ-Bpa | 349 |
| FEWTPGY-Bpa-QJY | 350 |
| AcFEWTPGY-Bpa-QJY | 351 |
| FEWTPG-Bpa-YQJY | 352 |
| AcFEWTPG-Bpa-YQJY | 353 |
| AcFE-Bpa-TPGYYQJY | 354 |
| AcFE-Bpa-TPGYYQJY | 355 |
| Bpa-EWTPGYYQJY | 356 |
| AcBpa-EWTPGYYQJY | 357 |
| VYWQPYSVQ | 358 |
| RLVYWQPYSVQR | 359 |
| RLVY-Nap-QPYSVQR | 360 |
| RLDYWQPYSVQR | 361 |
| RLVWFQPYSVQR | 362 |
| RLVYWQPYSIQR | 363 |
| DNSSWYDSFLL | 364 |
| DNTAWYESFLA | 365 |
| DNTAWYENFLL | 366 |
| PARE DNTAWYDSFLI WC | 367 |
| TSEY DNTTWYEKFLA SQ | 368 |
| SQIP DNTAWYQSFLL HG | 369 |
| SPFI DNTAWYENFLL TY | 370 |
| EQIY DNTAWYDHFLL SY | 371 |
| TPFI DNTAWYENFLL TY | 372 |
| TYTY DNTAWYERFLM SY | 373 |
| TMTQ DNTAWYENFLL SY | 374 |
| TI DNTAWYANLVQ TYPQ | 375 |
| TI DNTAWYERFLA QYPD | 376 |
| HI DNTAWYENFLL TYTP | 377 |
| SQ DNTAWYENFLL SYKA | 378 |
| QI DNTAWYERFLL QYNA | 379 |
| NQ DNTAWYESFLL QYNT | 380 |
| TI DNTAWYENFLL NHNL | 381 |
| HY DNTAWYERFLQ QGWH | 382 |
| ETPFTWEESNAYYWQPYALPL | 383 |
| YIPFTWEESNAYYWQPYALPL | 384 |
| DGYDRWRQSGERYWQPYALPL | 385 |
| pY-INap-pY-QJYALPL | 386 |
| TANVSSFEWTPGYWQPYALPL | 387 |
| FEWTPGYWQJYALPL | 388 |
| FEWTPGYWQPYALPLSD | 389 |
| FEWTPGYYQJYALPL | 390 |
| FEWTPGYWQJY | 391 |
| AcFEWTPGYWQJY | 392 |
| AcFEWTPGWYQJY | 393 |
| AcFEWTPGYYQJY | 394 |
| AcFEWTPaYWQJY | 395 |
| AcFEWTPaWYQJY | 396 |
| AcFEWTPaYYQJY | 397 |
| FEWTPGYYQJYALPL | 398 |
| FEWTPGYWQJYALPL | 399 |
| FEWTPGWYQJYALPL | 400 |
| TANVSSFEWTPGYWQPYALPL | 401 |
| AcFEWTPGYWQJY | 402 |
| AcFEWTPGWYQJY | 403 |
| AcFEWTPGYYQJY | 404 |
| AcFEWTPAYWQJY | 405 |
| AcFEWTPAWYQJY | 406 |
| AcFEWTPAYYQJY | 407 |

The peptides above correspond to the peptides of Table 4 and SEQ ID NOS: 212, 907-910, 917, 979, 213 to 271, 671 to 906, and 911 to 978, and 980 to 1023 (incorporated herein by reference) of the aforementioned U.S. Pat. No. 6,660,843 and may be prepared by methods known in the art. Such peptides are within the scope of IL-1 inhibitors in this invention.

Peptides such as those described in Table I may be used to make molecules of the formulae
(X¹)ₐ—F¹(X²)_{b} I
X¹—F¹ II
F¹—X² III
F¹-(L¹)_{c}-P¹ IV
F¹-(L¹)_{c}—P¹-(L²)_{d}-P² V
and multimers thereof wherein:

F¹ is a half-life extending vehicle, such as polyethylene glycol (PEG), dextran, or preferably an Fc domain;

X¹ and X² are each independently selected from -(L¹)_{c}-P¹, -(L¹)_{c}-P¹-(L²)_{d}-P², -(L¹)_{c}-P¹-(L²)_{d}-P²-(L³)ₑ-P³, and -(L¹)_{c}-P¹-(L²)_{d}-P²-(L³)ₑ-P³-(L⁴)_{f}-P⁴

P¹, P², P³, and P⁴ are each independently sequences of pharmacologically active IL-1 antagonist peptides;

L¹, L², L³, and L⁴ are each independently linkers; and

a, b, c, d, e, and f are each independently 0 or 1, provided that at least one of a and b is 1.

Molecules of the foregoing formulae in which F¹ is an Fc domain have been named “peptibodies.” Peptibodies may be prepared as described in the aforementioned U.S. Pat. No. 6,660,843. All vehicle-linked peptide molecules, including peptibodies, are IL-1 inhibitors within the meaning of this specification.

Soluble IL-1 Receptors

“Soluble IL-1 receptor molecules” refers to soluble IL-1 RI (sIL-1 RI), soluble IL-1 RII (sIL-1 RII), and soluble IL-1 RacP (sIL-1 RacP); fragments of sIL-1 RI, sIL-1 RII, and sIL-1RacP; and fusion proteins of sIL-1 RI, sIL-1 RII, sIL-1 RacP and fragments of any thereof, including “IL-1 trap” molecules and fusion proteins with human serum albumin, transthyretin or an Fc domain; and derivatives of any of the foregoing (e.g., soluble receptor linked to polyethylene glycol). Soluble IL-1 receptor molecules are described in U.S. Pat. Nos. 5,492,888; 5,488,032; 5,464,937; 5,319,071; and 5,180,812, the disclosures of which are hereby incorporated by reference.

Fragments of the IL-1 receptor include, but are not limited to, synthetic polypeptides corresponding to residues 86-93 of the human type I IL-1 receptor, which bind IL-1α and β and inhibit IL-1 activity in vitro and in vivo. See Tanihara et al. (1992) Biochem. Biophys. Res. Commun. 188: 912.

IL-1 Trap

The IL-1 trap is as essentially described in U.S. Pat. No. 5,844,099, which is hereby incorporated by reference. Briefly, the IL-1 trap is a fusion protein comprising the human cytokine receptor extracellular domains and the Fc portion of human IgG1. The IL-1 trap incorporates into a single molecule the extracellular domains of both receptor components required for IL-1 signaling; the IL-1 Type I receptor (IL-1 RI) and the IL-1 receptor accessory protein (AcP). Since it contains both receptor components, the IL-1 trap binds IL-1α and IL-1β with picomolar affinity, while the IL-1R1 alone in the absence of AcP binds with about 1 nM affinity. The IL-1 trap was created by fusing the sequences encoding the extracellular domains of the AcP, IL-1RI, and Fc in line without any intervening linker sequences. An expression construct encoding the fusion protein is transfected into Chinese hamster ovary (CHO) cells, and high producing lines are isolated that secrete the IL-1 trap into the medium. The IL-1 TRAP is a dimeric glycoprotein with a protein molecular weight of 201 kD and including glycosylation has a total molecular weight of .about.252 kD. Disulfide bonds in the Fc region covalently link the dimer.

Vascular Calcification

“Vascular calcification,” as used herein, means formation, growth or deposition of extracellular matrix hydroxyapatite (calcium phosphate) crystal deposits in blood vessels. Vascular calcification encompasses coronary, valvular, aortic, and other blood vessel calcification. The term includes atherosclerotic and medial wall calcification.

“Atherosclerotic calcification” means vascular calcification occurring in atheromatous plaques along the intimal layer of arteries.

“Medial calcification,” “medial wall calcification,” or “Monckeberg's sclerosis,” as used herein, means calcification characterized by the presence of calcium in the medial wall of arteries.

“Inhibiting,” in connection with inhibiting vascular calcification, is intended to mean preventing, retarding, or reversing formation, growth or deposition of extracellular matrix hydroxyapatite crystal deposits.

The term “treatment” or “treating” includes the administration, to a person in need, of an amount of an IL-1 inhibitor, which will inhibit or reverse development of a pathological vascular calcification condition.

The term “prevention” or “preventing” includes either preventing the onset or preventing/slowing the progression of clinically evident vascular calcification disorders altogether or preventing the onset of a preclinically evident stage of vascular calcification disorder in individuals. This includes prophylacetic treatment of those at risk of developing a vascular calcification disorder.

The phrase “therapeutically effective amount” is the amount of the IL-1 inhibitor that will achieve the goal of improvement in disorder severity and the frequency of incidence. The improvement in disorder severity includes the reversal of vascular calcification, as well as slowing down the progression of vascular calcification. In one aspect, “therapeutically effective amount” means the amount of the IL-1 inhibitor that decreases serum creatinine levels or prevents an increase in serum creatinine levels.

As used herein, the term “subject” is intended to mean a human or other mammal, exhibiting, or at risk of developing, calcification. Such an individual can have, or be at risk of developing, for example, vascular calcification associated with conditions such as atherosclerosis, stenosis, restenosis, renal failure, diabetes, prosthesis implantation, tissue injury or age-related vascular disease. The prognostic and clinical indications of these conditions are known in the art. An individual treated by a method of the invention can have a systemic mineral imbalance associated with, for example, diabetes, chronic kidney disease, renal failure, kidney transplantation or kidney dialysis.

Animal models that are reliable indicators of human atherosclerosis, renal failure, hyperphosphatemia, diabetes, age-related vascular calcification and other conditions associated with vascular calcification are known in the art. For example, Yamaguchi et al., Exp. Path., describe an experimental model of calcification of the vessel wall. 25: 185-190, 1984.

Assessment of Vascular Calcification

Methods of detecting and measuring vascular calcification are well known in the art. In one aspect, methods of measuring calcification include direct methods of detecting and measuring extent of calcium-phosphorus depositions in blood vessels.

In one aspect, direct methods of measuring vascular calcification comprise in vivo imaging methods such as plain film roentgenography, coronary arteriography; fluoroscopy, including digital subtraction fluoroscopy; cinefluorography; conventional, helical, and electron beam computed tomography; intravascular ultrasound (IVUS); magnetic resonance imaging; and transthoracic and transesophageal echocardiography. Persons skilled in the art most commonly use fluoroscopy and EBCT to detect calcification noninvasively. Coronary interventionalists use cinefluorography and IVUS to evaluate calcification in specific lesions before angioplasty.

In one aspect, vascular calcification can be detected by plain film roentgenography. The advantage of this method is availability of the film and the low cost of the method, however, the disadvantage is its low sensitivity. Kelley M. & Newell J. Cardiol Clin. 1: 575-595, 1983.

In another aspect, fluoroscopy can be used to detect calcification in coronary arteries. Although fluoroscopy can detect moderate to large calcifications, its ability to identify small calcific deposits is low. Loecker et al. J. Am. Coll. Cardiol. 19: 1167-1172, 1992. Fluoroscopy is widely available in both inpatient and outpatient settings and is relatively inexpensive, but it has several disadvantages. In addition to only a low to moderate sensitivity, fluoroscopic detection of calcium is dependent on the skill and experience of the operator as well as the number of views studied. Other important factors include variability of fluoroscopic equipment, the patient's body habitus, overlying anatomic structures, and overlying calcifications in structures such as vertebrae and valve annuli. With fluoroscopy, quantification of calcium is not possible, and film documentation is not commonly obtained.

In yet another aspect, vascular detection can be detected by conventional computed tomography (CT). Because calcium attenuates the x-ray beam, computed tomography (CT) is extremely sensitive in detecting vascular calcification. While conventional CT appears to have better capability than fluoroscopy to detect coronary artery calcification, its limitations are slow scan times resulting in motion artifacts, volume averaging, breathing misregistration, and inability to quantify amount of plaque. Wexler et al. Circulation 94: 1175-1192, 1996.

In a further aspect, calcification can be detected by helical or spiral computer tomography, which has considerably faster scan times than conventional CT. Overlapping sections also improve calcium detection. Shemesh et al. reported coronary calcium imaging by helical CT as having a sensitivity of 91% and a specificity of 52% when compared with angiographically significant coronary obstructive disease. Shemesh et al. Radiology 197: 779-783, 1995. However, other preliminary data have shown that even at these accelerated scan times, and especially with single helical CT, calcific deposits are blurred due to cardiac motion, and small calcifications may not be seen. Baskin et al. Circulation 92(suppl I): 1-651, 1995. Thus, helical CT remains superior to fluoroscopy and conventional CT in detecting calcification. Double-helix CT scanners appear to be more sensitive than single-helix scanners in detection of coronary calcification because of their higher resolution and thinner slice capabilities. Wexler et al., supra.

In another aspect, Electron Beam Computed Tomography (EBCT) can be used for detection of vascular calcification. EBCT uses an electron gun and a stationary tungsten “target” rather than a standard x-ray tube to generate x-rays, permitting very rapid scanning times. Originally referred to as cine or ultrafast CT, the term EBCT is now used to distinguish it from standard CT scans because modern spiral scanners are also achieving subsecond scanning times. For purposes of detecting coronary calcium, EBCT images are obtained in 100 ms with a scan slice thickness of 3 mm. Thirty to 40 adjacent axial scans are obtained by table incrementation. The scans, which are usually acquired during one or two separate breath-holding sequences, are triggered by the electrocardiographic signal at 80% of the RR interval, near the end of diastole and before atrial contraction, to minimize the effect of cardiac motion. The rapid image acquisition time virtually eliminates motion artifact related to cardiac contraction. The unopacified coronary arteries are easily identified by EBCT because the lower CT density of periarterial fat produces marked contrast to blood in the coronary arteries, while the mural calcium is evident because of its high CT density relative to blood. Additionally, the scanner software allows quantification of calcium area and density. An arbitrary scoring system has been devised based on the x-ray attenuation coefficient, or CT number measured in Hounsfield units, and the area of calcified deposits. Agatston et al. J. Am. Coll. Cardiol. 15:827-832, 1990. A screening study for coronary calcium can be completed within 10 or 15 minutes, requiring only a few seconds of scanning time. Electron beam CT scanners are more expensive than conventional or spiral CT scanners and are available in relatively fewer sites.

In one aspect, intravascular ultrasound (IVUS) can be used for detecting vascular calcification, in particular, coronary atherosclerosis. Waller et al. Circulation 85: 2305-2310, 1992. By using transducers with rotating reflectors mounted on the tips of catheters, it is possible to obtain cross-sectional images of the coronary arteries during cardiac catheterization. The sonograms provide information not only about the lumen of the artery but also about the thickness and tissue characteristics of the arterial wall. Calcification is seen as a hyperechoic area with shadowing: fibrotic noncalcified plaques are seen as hyperechoic areas without shadowing. Honye et al. Trends Cardiovasc Med. 1: 305-311, 1991. The disadvantages in use of IVUS, as opposed to other imaging modalities, are that it is invasive and currently performed only in conjunction with selective coronary angiography, and it visualizes only a limited portion of the coronary tree. Although invasive, the technique is clinically important because it can show atherosclerotic involvement in patients with normal findings on coronary arteriograms and helps define the morphological characteristics of stenotic lesions before balloon angioplasty and selection of atherectomy devices. Tuzcu et al. J. Am. Coll. Cardiol. 27: 832-838, 1996.

In another aspect, vascular calcification can be measured by magnetic resonance imaging (MRI). However, the ability of MRI to detect coronary calcification is somewhat limited. Because microcalcifications do not substantially alter the signal intensity of voxels that contain a large amount of soft tissue, the net contrast in such calcium collections is low. Therefore, MRI detection of small quantities of calcification is difficult, and there are no reports or expected roles for MRI in detection of coronary artery calcification. Wexler et al., supra.

In another aspect, vascular calcification can be measured by transthoracic (surface) echocardiography, which is particularly sensitive to detection of mitral and aortic valvular calcification; however, visualization of the coronary arteries has been documented only on rare occasions because of the limited available external acoustic windows. Transesophageal echocardiography is a widely available methodology that often can visualize the proximal coronary arteries. Koh et al. Int. J. Cardiol. 43: 202-206, 1994. Fernandes et al. Circulation 88: 2532-2540, 1993.

In another aspect, vascular calcification can be assessed ex vivo by Van Kossa method. This method relies upon the principle that silver ions can be displaced from solution by carbonate or phosphate ions due to their respective positions in the electrochemical series. The argentaffin reaction is photochemical in nature and the activation energy is supplied from strong visible or ultra-violet light. Since the demonstrable forms of tissue carbonate or phosphate ions are invariably associated with calcium ions the method may be considered as demonstrating sites of tissue calcium deposition.

Other methods of direct measuring calcification may include, but not limited to, immunofluorescent staining and densitometry. In another aspect, methods of assessing vascular calcification include methods of measuring determinants and/or risk factors of vascular calcification. Such factors include, but are not limited to, serum levels of phosphorus, calcium, and calcium×phosphorus product, parathyroid hormone (PTH), low-density lipoprotein cholesterol (LDL), high-density lipoprotein cholesterol (HDL), triglycerides, and creatinine. Methods of measuring these factors are well known in the art. Other methods of assessing vascular calcification include assessing factors of bone formation. Such factors include bone formation markers such as bone-specific alkaline phosphatase (BSAP), osteocalcin (OC), carboxyterminal propeptide of type I collagen (PICP), and aminoterminal propeptide of type I collagen (PINP); serum bone resorption markers such as cross-linked C-telopeptide of type I collagen (ICTP), tartrate-resistant acid phosphatase, TRACP and TRAP5B, N-telopeptide of collagen cross-links (NTx), and C-telopeptide of collagen cross-links (CTx); and urine bone resorption markers, such as hydroxyproline, free and total pyridinolines (Pyd), free and total deoxypyridinolines (Dpd), N-telopeptide of collagen cross-links (NTx), and C-telopeptide of collagen cross-links (CTx).

Methods of Treatment

In one aspect, the invention provides a method of inhibiting, decreasing or preventing vascular calcification in an individual. The method comprises administering to the individual a therapeutically effective amount of the IL-1 inhibitor of the invention. In one aspect, administration of the compound of the invention retards or reverses the formation, growth or deposition of extracellular matrix hydroxyapatite crystal deposits. In another aspect of the invention, administration of the compound of the invention prevents the formation, growth or deposition of extracellular matrix hydroxyapatite crystal deposits.

Methods of the invention may be used to prevent or treat atherosclerotic calcification and medial calcification and other conditions characterized by vascular calcification. In one aspect, vascular calcification may be associated with chronic renal insufficiency or end-stage renal disease. In another aspect, vascular calcification may be associated with pre- or post-dialysis or uremia. In a further aspect, vascular calcification may be associated with diabetes mellitus I or II. In yet another aspect, vascular calcification may be associated with a cardiovascular disorder.

In one aspect, administration of an effective amount of an IL-1 inhibitor can reduce serum PTH without causing aortic calcification. In another aspect, administration of an IL-1 inhibitor can reduce serum creatinine level or can prevent increase of serum creatinine level. In another aspect, administration of an IL-1 inhibitor can attenuates parathyroid (PT) hyperplasia.

In one aspect of combination therapy, the IL-1 inhibitors of the invention may be used with calcimimetics, vitamins and their analogs, such as vitamin D and analogs thereof (including vitamin D sterols such as calcitriol, alfacalcidol, doxercalciferol, maxacalcitol and paricalcitol), antibiotics, lanthanum carbonate, lipid-lowering agents, such as LIPITOR®, anti-hypertensives, anti-inflammatory agents (steroidal and non-steroidal), inhibitors of pro-inflammatory cytokine (ENBREL®, KINERET®), and cardiovascular agents. vitamin D sterols and/or RENAGEL®. In one aspect, the compositions of the invention may be administered before, concurrently, or after administration of calcimimetics, vitamin D sterols and/or RENAGEL®. The dosage regimen for treating a disease condition with the combination therapy of this invention is selected in accordance with a variety of factors, including the type, age, weight, sex and medical condition of the patient, the severity of the disease, the route of administration, and the particular compound employed, and thus may vary widely.

In accordance with this invention, IL-1 inhibitors may be administered alone or in combination with other drugs for treating vascular calcification, such as vitamin D sterols and/or RENAGEL®. Vitamin D sterols can include calcitriol, alfacalcidol, doxercalciferol, maxacalcitol or paricalcitol. In one aspect, IL-1 inhibitors can be administered before or after administration of vitamin D sterols. In another aspect, IL-1 inhibitors can be co-administered with vitamin D sterols. The methods of the invention can be practiced to attenuate the mineralizing effect of calcitriol on vascular tissue. In one aspect, the methods of the invention can be used to reverse the effect of calcitriol of increasing the serum levels of calcium, phosphorus and Ca×P product thereby preventing or inhibiting vascular calcification. In another aspect, the methods of the invention can be used to stabilize or decrease serum creatinine levels. In one aspect, in addition to creatinine level increase due to a disease, a further increase in creatinine level can be due to treatment with vitamin D sterols such as calcitriol.

In addition, IL-1 inhibitors may be administered in conjunction with surgical and non-surgical treatments. In one aspect, the methods of the invention can be practiced in injunction with dialysis.

The following examples are offered to more fully illustrate the invention, but are not to be construed as limiting the scope thereof.

### EXAMPLE 1

### Adenine-Induced Secondary Hyperparathyroidism (SHPT) and Calcification in Rats and Prevention of Aortic Vascular Calcification by Fc-IL-1ra

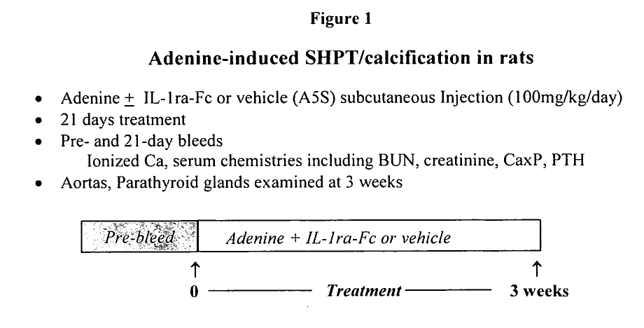

This experiment used the protocol shown in FIG. 1.

Adenine, included as a dietary supplement (0.75%), was fed to adult, male Sprague-Dawley rats. Blood for chemistry analyses (total serum calcium, phosphorous, blood urea nitrogen [BUN], creatinine, PTH) was collected before and again on drug treatment days 0 (pretreatment) and 21 from the retro-orbital sinus of anesthetized rats. Blood (0.5 ml) was collected for PTH levels into SST (clot activator) brand blood tubes and allowed to clot. Serum was removed and stored at −70° C. until assayed. PTH levels were quantified according to the vendor's instructions using rat PTH (1-34) immunoradiometric assay kit (Immutopics, San Clemente, Calif.). Calcium and phosphorous were measured using a blood chemistry analyzer (AU 400; Olympus, Melville, N.Y.).

Vascular calcification was assessed by quantifying the bone mineral density from fixed (formalin, PBS), isolated aortas using a Dxa scanner (Piximus densitomer, GE Healthcare).

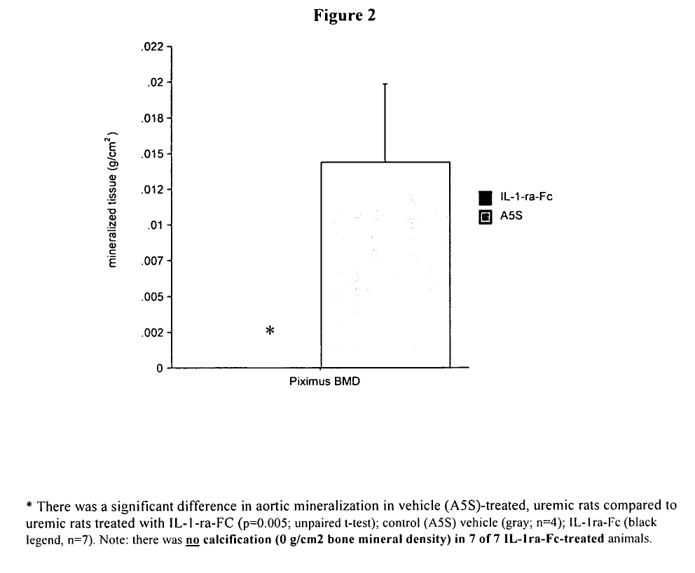

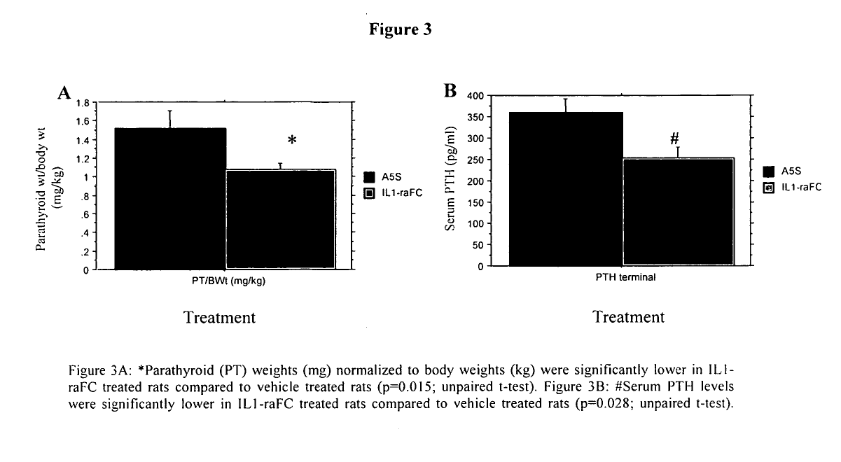

In this model, CKD/SHPT induced by dietary adenine leads to significant renal impairment (increased BUN, creatinine), and aortic vascular calcification. Fc-IL-1ra prevented the development of aortic vascular calcification (decreased bone mineral density content of the aorta) in this model (FIG. 2), but did not affect BUN, creatinine levels. In addition, Fc-IL-1ra reduced the size of the parathyroid gland in this model (FIG. 3A) and decreased serum PTH levels (FIG. 3B).

### EXAMPLE 2

### Effect of IL-1ra on Vitamin D-Induced Vascular Calcification

The protocol used in this experiment may be summarized as follows.

PILOT: IL-1ra effects on vascular calcification.

Vitamin D 100 ng×3 weeks±IL-1 Ra (subcutaneous)→aortas for VC

Female, Lewis rats 250 g (pump implantation)

Vitamin D₃, supplied as 1α, 25-dihydroxycholecalciferol from Sigma-Aldrich, Corp (St. Louis, Mo.), was dissolved in 90% ethanol to create a 1 mM stock solution that was stored at −20° C. until final dilution in phosphate buffered saline (PBS). Vitamin D₃ (0.1 μg, in a dose volume of 0.2 ml PBS) was administered by subcutaneous (s.c.) injection.

IL-1ra

Dose 5 mg/kg/h SC infusion

Endpoint: Von Kossa

Groups (n=6/group)

1. Vitamin D 100 ng

2. Vitamin D 100 ng+IL1-Ra (5 mg/kg/h SC)

3. Vitamin D 100 ng+vehicle (pump)

4. vehicle (n=2)

21 days treatment

Sacrifice: remove aortas for von Kossa staining

### EXAMPLE 3

### Effect of Fc-IL-1ra on Vitamin D-Induced Vascular Calcification

The protocol used in this experiment may be summarized as follows.

PILOT: Fc IL-1ra effects on vascular calcification

Vitamin D 100 ng×3 weeks±Fc IL-1ra (subcutaneous)→aortas for VC

Female, Lewis rats 250 g and/or male, SD rats (250 g)

Vitamin D₃, supplied as 1α, 25-dihydroxycholecalciferol from Sigma-Aldrich, Corp (D-1530-0.1 mg, St. Louis, Mo.), was dissolved in 90% ethanol to create a 1 mM stock solution that was stored at −20° C. until final dilution in phosphate buffered saline (PBS). Vitamin D₃ (0.1 μg, in a dose volume of 0.2 ml PBS) was administered by subcutaneous (s.c.) injection.

Fc IL-1ra

Dose 100 mg/kg SC/day

Endpoint: Von Kossa

Groups (n=6/group)

1. Vitamin D 100 ng

2. Vitamin D 100 ng+Fc IL1-Ra (100 mg/kg/d SC)

3. Vitamin D 100 ng+vehicle (pump)

4. vehicle (n=2)

21 days treatment

Sacrifice: remove aortas for von Kossa staining

### EXAMPLE 4

### Attenuation of Calcitriol (Vitamin D3)-Induced Aortic Vascular Calcification with IL-1ra-Fc (Inhibitor of IL-1)

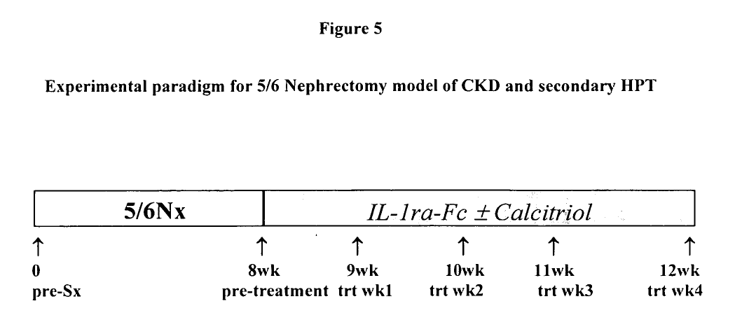

We administered the IL-1 receptor antagonist Fc-IL-1ra, calcitriol, the combination of Fc-IL-1ra+calcitriol) or their corresponding vehicles to a rodent animal model of chronic kidney disease (CKD) and secondary hyperparathyroidism (SHPT) induced by subtotal nephrectomy (5/6Nx). The experimental paradigm is shown in FIG. 5, with the treatment groups set out in Table 2 below.

TABLE 2
Treatment groups (n = 10/group): 5/6 nx, male, SD rats (n = 45)

| | |
|---|---|
| FC-IL-1ra 100 mg/kg/day s.c. in A5S buffer | n = 10 at week 4 |
| Vehicle (A5S buffer; 1 ml/kg/day s.c) | n = 10 at week 4 |
| FC-IL-1ra 100 mg/kg/day s.c. + Calcitriol (30 ng) | n = 10 at week 4 |
| Calcitriol (30 ng) | n = 10 at week 4 |
| Calcitriol Vehicle | n = 5 |

Aortas were removed at treatment week 4 (trt wk4), fixed and stained for mineralization (Von Kossa) and the severity determined and scored by a pathologist blinded to the treatments (Calcification Scores: 0=no calcification; 1=minimal; 2=mild; 3=moderate; 4=marked; 5=severe).

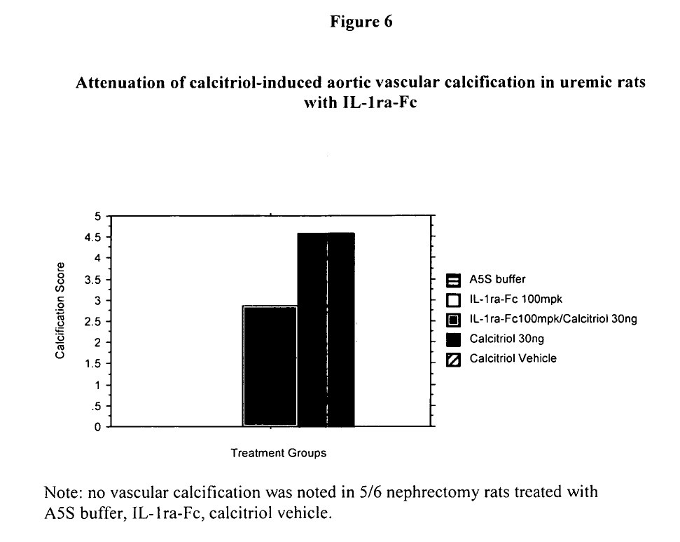

Fc-IL-1ra administered systemically to a rodent animal model of established chronic kidney disease accompanied with secondary hyperparathyroidism (induced by subtotal (5/6) nephrectomy) did not cause vascular calcification, whereas calcitriol caused marked to severe aortic calcification. Fc-IL-1ra attenuated calcitriol (Vitamin D3)-induced aortic vascular calcification, in this model whereby uremia was established for 8 weeks before treatments were started (FIG. 6). In addition, the incidence of severe calcification in calcitriol-treated uremic subjects (4/8 or 50%) was reduced in animals receiving calcitriol in combination with Fc-IL-1ra (1/8 or 12.5%).

The foregoing specification includes numerous definitions. These definitions apply to the terms as used throughout this specification, unless otherwise limited in specific instances. Definitions apply equally to the plural and singular forms of each term.

All publications, patents and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of inhibiting, decreasing, or preventing vascular calcification in a subject comprising administering a therapeutically effective amount of an IL-1 inhibitor to the subject.

2. The method of claim 1, wherein the subject is suffering from chronic renal insufficiency.

3. The method of claim 1, wherein the subject is suffering from end-stage renal disease.

4. The method of claim 1, wherein the subject is pre-dialysis.

5. The method of claim 1, wherein the subject is suffering from uremia.

6. The method of claim 1, wherein the subject is suffering from diabetes mellitus I or II.

7. The method of claim 1, wherein the subject has a cardiovascular disorder.

8. The method of claim 1, wherein the IL-1 inhibitor is or comprises the sequence of anakinra.

9. The method of claim 1, wherein the IL-1 inhibitor is an IL-1RI antibody.

10. The method of claim 1, wherein the IL-1 inhibitor is administered in combination with a calcimimetic compound.

11. The method of claim 1, wherein the IL-1 inhibitor is administered in combination with cinacalcet HCL.

12. The method of claim 1, wherein the IL-1 inhibitor is administered in combination with a vitamin D sterol.

13. The method of claim 1, wherein the IL-1 inhibitor is administered in combination with RENAGEL®.

14. A method of decreasing serum creatinine levels in a subject, comprising administering a therapeutically effective amount of an IL-1 inhibitor to the subject.

15. The method of claim 14, wherein the subject is suffering from increased serum creatinine levels induced by the administration of a vitamin D sterol to the subject.


SEQUENCE LISTING (412 sequences)

SEQ ID NO: 1 - protein, 380 aa, Homo sapiens
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 2 - protein, 21 aa, Artificial Sequence
  IL-1 Antagonist Peptide
TANVSSFEWTPYYWQPYALPL

SEQ ID NO: 3 - protein, 18 aa, Artificial Sequence
  IL-1 Antagonist Peptide
SWTDYGYWQPYALPISGL

SEQ ID NO: 4 - protein, 21 aa, Artificial Sequence
  IL-1 Antagonist Peptide
ETPFTWEESNAYYWQPYALPL

SEQ ID NO: 5 - protein, 21 aa, Artificial Sequence
  IL-1 Antagonist Peptide
ENTYSPNWADSMYWQPYALPL

SEQ ID NO: 6 - protein, 21 aa, Artificial Sequence
  IL-1 Antagonist Peptide
SVGEDHNFWTSEYWQPYALPL

SEQ ID NO: 7 - protein, 21 aa, Artificial Sequence
  IL-1 Antagonist Peptide
DGYDRWRQSGERYWQPYALPL

SEQ ID NO: 8 - protein, 11 aa, Artificial Sequence
  IL-1 Antagonist Peptide
FEWTPGYWQPY

SEQ ID NO: 9 - protein, 11 aa, Artificial Sequence
  IL-1 Antagonist Peptide
FEWTPGYWQHY

SEQ ID NO: 10 - protein, 11 aa, Artificial Sequence
  IL-1 Antagonist Peptide
  misc_feature (10)..(10): Xaa can be any naturally occurring amino acid
FEWTPGWYQXY

SEQ ID NO: 11 - protein, 13 aa, Artificial Sequence
  IL-1 Antagonist Peptide
  misc_feature (12)..(12): Xaa can be any naturally occurring amino acid
ACFEWTPGWYQXY

SEQ ID NO: 12 - protein, 12 aa, Artificial Sequence
  IL-1 Antagonist Peptide
  misc_feature (11)..(11): Xaa can be any naturally occurring amino acid
FEWTPGWPYQXY

SEQ ID NO: 13 - protein, 11 aa, Artificial Sequence
  IL-1 Antagonist Peptide
  misc_feature (10)..(10): Xaa can be any naturally occurring amino acid
FAWTPGYWQXY

SEQ ID NO: 14 - protein, 11 aa, Artificial Sequence
  IL-1 Antagonist Peptide
  misc_feature (10)..(10): Xaa can be any naturally occurring amino acid
FEWAPGYWQXY

SEQ ID NO: 15 - protein, 11 aa, Artificial Sequence
  IL-1 Antagonist Peptide
  misc_feature (10)..(10): Xaa can be any naturally occurring amino acid
FEWVPGYWQXY

SEQ ID NO: 16 - protein, 11 aa, Artificial Sequence
  IL-1 Antagonist Peptide
  misc_feature (10)..(10): Xaa can be any naturally occurring amino acid
FEWTPGYWQXY

SEQ ID NO: 17 - protein, 13 aa, Artificial Sequence
  IL-1 Antagonist Peptide
  misc_feature (12)..(12): Xaa can be any naturally occurring amino acid
ACFEWTPGYWQXY

SEQ ID NO: 18 - protein, 11 aa, Artificial Sequence
  IL-1 Antagonist Peptide
  misc_feature (10)..(10): Xaa can be any naturally occurring amino acid
FEWTPAWYQXY

SEQ ID NO: 19 - protein, 13 aa, Artificial Sequence
  IL-1 Antagonist Peptide
  misc_feature (12)..(12): Xaa can be any naturally occurring amino acid
FEWTPSARWYQXY

SEQ ID NO: 20 - protein, 11 aa, Artificial Sequence
  IL-1 Antagonist Peptide
FEWTPGYYQPY

SEQ ID NO: 21 - protein, 11 aa, Artificial Sequence
  IL-1 Antagonist Peptide
FEWTPGWWQPY

SEQ ID NO: 22 - protein, 11 aa, Artificial Sequence
  IL-1 Antagonist Peptide
FEWTPNYWQPY

SEQ ID NO: 23 - protein, 11 aa, Artificial Sequence
  IL-1 Antagonist Peptide
  misc_feature (10)..(10): Xaa can be any naturally occurring amino acid
FEWTPVYWQXY

SEQ ID NO: 24 - protein, 13 aa, Artificial Sequence
  IL-1 Antagonist Peptide
  misc_feature (12)..(12): Xaa can be any naturally occurring amino acid
FEWTPECGYWQXY

SEQ ID NO: 25 - protein, 13 aa, Artificial Sequence
  IL-1 Antagonist Peptide
  misc_feature (12)..(12): Xaa can be any naturally occurring amino acid
FEWTPAIBYWQXY

SEQ ID NO: 26 - protein, 13 aa, Artificial Sequence
  IL-1 Antagonist Peptide
  misc_feature (12)..(12): Xaa can be any naturally occurring amino acid
FEWTSARGYWQXY

SEQ ID NO: 27 - protein, 11 aa, Artificial Sequence
  IL-1 Antagonist Peptide
FEWTPGYWQPY

SEQ ID NO: 28 - protein, 11 aa, Artificial Sequence
  IL-1 Antagonist Peptide
FEWTPGYWQHY

SEQ ID NO: 29 - protein, 11 aa, Artificial Sequence
  IL-1 Antagonist Peptide
  misc_feature (10)..(10): Xaa can be any naturally occurring amino acid
FEWTPGWYQXY

SEQ ID NO: 30 - protein, 13 aa, Artificial Sequence
  IL-1 Antagonist Peptide
  misc_feature (12)..(12): Xaa can be any naturally occurring amino acid
ACFEWTPGWYQXY

SEQ ID NO: 31 - protein, 12 aa, Artificial Sequence
  IL-1 Antagonist Peptide
  misc_feature (11)..(11): Xaa can be any naturally occurring amino acid
FEWTPGWPYQXY

SEQ ID NO: 32 - protein, 11 aa, Artificial Sequence
  IL-1 Antagonist Peptide
  misc_feature (10)..(10): Xaa can be any naturally occurring amino acid
FAWTPGYWQXY

SEQ ID NO: 33 - protein, 11 aa, Artificial Sequence
  IL-1 Antagonist Peptide
  misc_feature (10)..(10): Xaa can be any naturally occurring amino acid
FEWAPGYWQXY

SEQ ID NO: 34 - protein, 11 aa, Artificial Sequence
  IL-1 Antagonist Peptide
  misc_feature (10)..(10): Xaa can be any naturally occurring amino acid
FEWVPGYWQXY

SEQ ID NO: 35 - protein, 11 aa, Artificial Sequence
  IL-1 Antagonist Peptide
  misc_feature (10)..(10): Xaa can be any naturally occurring amino acid
FEWTPGYWQXY

SEQ ID NO: 36 - protein, 13 aa, Artificial Sequence
  IL-1 Antagonist Peptide
  misc_feature (12)..(12): Xaa can be any naturally occurring amino acid
ACFEWTPGYWQXY

SEQ ID NO: 37 - protein, 11 aa, Artificial Sequence
  IL-1 Antagonist Peptide
  misc_feature (10)..(10): Xaa can be any naturally occurring amino acid
FEWTPAWYQXY

SEQ ID NO: 38 - protein, 13 aa, Artificial Sequence
  IL-1 Antagonist Peptide
  misc_feature (12)..(12): Xaa can be any naturally occurring amino acid
FEWTPSARWYQXY

SEQ ID NO: 39 - protein, 11 aa, Artificial Sequence
  IL-1 Antagonist Peptide
FEWTPGYYQPY

SEQ ID NO: 40 - protein, 11 aa, Artificial Sequence
  IL-1 Antagonist Peptide
FEWTPGWWQPY

SEQ ID NO: 41 - protein, 11 aa, Artificial Sequence
  IL-1 Antagonist Peptide
FEWTPNYWQPY

SEQ ID NO: 42 - protein, 11 aa, Artificial Sequence
  IL-1 Antagonist Peptide
  misc_feature (10)..(10): Xaa can be any naturally occurring amino acid
FEWTPVYWQXY

SEQ ID NO: 43 - protein, 13 aa, Artificial Sequence
  IL-1 Antagonist Peptide
  misc_feature (12)..(12): Xaa can be any naturally occurring amino acid
FEWTPECGYWQXY

SEQ ID NO: 44 - protein, 13 aa, Artificial Sequence
  IL-1 Antagonist Peptide
  misc_feature (12)..(12): Xaa can be any naturally occurring amino acid
FEWTPAIBYWQXY

SEQ ID NO: 45 - protein, 13 aa, Artificial Sequence
  IL-1 Antagonist Peptide
  misc_feature (12)..(12): Xaa can be any naturally occurring amino acid
FEWTSARGYWQXY

SEQ ID NO: 46 - protein, 15 aa, Artificial Sequence
  IL-1 Antagonist Peptide
FEWTPGYWQPYALPL

SEQ ID NO: 47 - protein, 13 aa, Artificial Sequence
  IL-1 Antagonist Peptide
  misc_feature (12)..(12): Xaa can be any naturally occurring amino acid
NAPEWTPGYYQXY

SEQ ID NO: 48 - protein, 11 aa, Artificial Sequence
  IL-1 Antagonist Peptide
  misc_feature (10)..(10): Xaa can be any naturally occurring amino acid
YEWTPGYYQXY

SEQ ID NO: 49 - protein, 11 aa, Artificial Sequence
  IL-1 Antagonist Peptide
  misc_feature (10)..(10): Xaa can be any naturally occurring amino acid
FEWVPGYYQXY

SEQ ID NO: 50 - protein, 11 aa, Artificial Sequence
  IL-1 Antagonist Peptide
  misc_feature (10)..(10): Xaa can be any naturally occurring amino acid
FEWTPSYYQXY

SEQ ID NO: 51 - protein, 11 aa, Artificial Sequence
  IL-1 Antagonist Peptide
  misc_feature (10)..(10): Xaa can be any naturally occurring amino acid
FEWTPNYYQXY

SEQ ID NO: 52 - protein, 4 aa, Artificial Sequence
  IL-1 Antagonist Peptide
TKPR

SEQ ID NO: 53 - protein, 5 aa, Artificial Sequence
  IL-1 Antagonist Peptide
RKSSK

SEQ ID NO: 54 - protein, 5 aa, Artificial Sequence
  IL-1 Antagonist Peptide
RKQDK

SEQ ID NO: 55 - protein, 6 aa, Artificial Sequence
  IL-1 Antagonist Peptide
NRKQDK

SEQ ID NO: 56 - protein, 6 aa, Artificial Sequence
  IL-1 Antagonist Peptide
RKQDKR

SEQ ID NO: 57 - protein, 9 aa, Artificial Sequence
  IL-1 Antagonist Peptide
ENRKQDKRF

SEQ ID NO: 58 - protein, 6 aa, Artificial Sequence
  IL-1 Antagonist Peptide
VTKFYF

SEQ ID NO: 59 - protein, 5 aa, Artificial Sequence
  IL-1 Antagonist Peptide
VTKFY

SEQ ID NO: 60 - protein, 5 aa, Artificial Sequence
  IL-1 Antagonist Peptide
VTDFY

SEQ ID NO: 61 - protein, 11 aa, Artificial Sequence
  IL-1 Antagonist Peptide
SHLYWQPYSVQ

SEQ ID NO: 62 - protein, 12 aa, Artificial Sequence
  IL-1 Antagonist Peptide
TLVYWQPYSLQT

SEQ ID NO: 63 - protein, 12 aa, Artificial Sequence
  IL-1 Antagonist Peptide
RGDYWQPYSVQS

SEQ ID NO: 64 - protein, 12 aa, Artificial Sequence
  IL-1 Antagonist Peptide
VHVYWQPYSVQT

SEQ ID NO: 65 - protein, 12 aa, Artificial Sequence
  IL-1 Antagonist Peptide
RLVYWQPYSVQT

SEQ ID NO: 66 - protein, 12 aa, Artificial Sequence
  IL-1 Antagonist Peptide
SRVWFQPYSLQS

SEQ ID NO: 67 - protein, 12 aa, Artificial Sequence
  IL-1 Antagonist Peptide
NMVYWQPYSIQT

SEQ ID NO: 68 - protein, 12 aa, Artificial Sequence
  IL-1 Antagonist Peptide
SVVFWQPYSVQT

SEQ ID NO: 69 - protein, 12 aa, Artificial Sequence
  IL-1 Antagonist Peptide
TFVYWQPYALPL

SEQ ID NO: 70 - protein, 12 aa, Artificial Sequence
  IL-1 Antagonist Peptide
TLVYWQPYSIQR

SEQ ID NO: 71 - protein, 12 aa, Artificial Sequence
  IL-1 Antagonist Peptide
RLVYWQPYSVQR

SEQ ID NO: 72 - protein, 12 aa, Artificial Sequence
  IL-1 Antagonist Peptide
SPVFWQPYSIQI

SEQ ID NO: 73 - protein, 12 aa, Artificial Sequence
  IL-1 Antagonist Peptide
WIEWWQPYSVQS

SEQ ID NO: 74 - protein, 12 aa, Artificial Sequence
  IL-1 Antagonist Peptide
SLIYWQPYSLQM

SEQ ID NO: 75 - protein, 12 aa, Artificial Sequence
  IL-1 Antagonist Peptide
TRLYWQPYSVQR

SEQ ID NO: 76 - protein, 12 aa, Artificial Sequence
  IL-1 Antagonist Peptide
RCDYWQPYSVQT

SEQ ID NO: 77 - protein, 12 aa, Artificial Sequence
  IL-1 Antagonist Peptide
MRVFWQPYSVQN

SEQ ID NO: 78 - protein, 12 aa, Artificial Sequence
  IL-1 Antagonist Peptide
KIVYWQPYSVQT

SEQ ID NO: 79 - protein, 12 aa, Artificial Sequence
  IL-1 Antagonist Peptide
RHLYWQPYSVQR

SEQ ID NO: 80 - protein, 12 aa, Artificial Sequence
  IL-1 Antagonist Peptide
ALVWWQPYSEQI

SEQ ID NO: 81 - protein, 12 aa, Artificial Sequence
  IL-1 Antagonist Peptide
SRVWFQPYSLQS

SEQ ID NO: 82 - protein, 10 aa, Artificial Sequence
  IL-1 Antagonist Peptide
WEQPYALPLE

SEQ ID NO: 83 - protein, 12 aa, Artificial Sequence
  IL-1 Antagonist Peptide
QLVWWQPYSVQR

SEQ ID NO: 84 - protein, 12 aa, Artificial Sequence
  IL-1 Antagonist Peptide
DLRYWQPYSVQV

SEQ ID NO: 85 - protein, 12 aa, Artificial Sequence
  IL-1 Antagonist Peptide
ELVWWQPYSLQL

SEQ ID NO: 86 - protein, 12 aa, Artificial Sequence
  IL-1 Antagonist Peptide
DLVWWQPYSVQW

SEQ ID NO: 87 - protein, 12 aa, Artificial Sequence
  IL-1 Antagonist Peptide
NGNYWQPYSFQV

SEQ ID NO: 88 - protein, 12 aa, Artificial Sequence
  IL-1 Antagonist Peptide
ELVYWQPYSIQR

SEQ ID NO: 89 - protein, 12 aa, Artificial Sequence
  IL-1 Antagonist Peptide
ELMYWQPYSVQE

SEQ ID NO: 90 - protein, 12 aa, Artificial Sequence
  IL-1 Antagonist Peptide
NLLYWQPYSMQD

SEQ ID NO: 91 - protein, 12 aa, Artificial Sequence
  IL-1 Antagonist Peptide
GYEWYQPYSVQR

SEQ ID NO: 92 - protein, 12 aa, Artificial Sequence
  IL-1 Antagonist Peptide
SRVWYQPYSVQR

SEQ ID NO: 93 - protein, 12 aa, Artificial Sequence
  IL-1 Antagonist Peptide
LSEQYQPYSVQR

SEQ ID NO: 94 - protein, 12 aa, Artificial Sequence
  IL-1 Antagonist Peptide
GGGWWQPYSVQR

SEQ ID NO: 95 - protein, 12 aa, Artificial Sequence
  IL-1 Antagonist Peptide
VGRWYQPYSVQR

SEQ ID NO: 96 - protein, 12 aa, Artificial Sequence
  IL-1 Antagonist Peptide
VHVYWQPYSVQR

SEQ ID NO: 97 - protein, 12 aa, Artificial Sequence
  IL-1 Antagonist Peptide
QARWYQPYSVQR

SEQ ID NO: 98 - protein, 12 aa, Artificial Sequence
  IL-1 Antagonist Peptide
VHVYWQPYSVQT

SEQ ID NO: 99 - protein, 12 aa, Artificial Sequence
  IL-1 Antagonist Peptide
RSVYWQPYSVQR

SEQ ID NO: 100 - protein, 12 aa, Artificial Sequence
  IL-1 Antagonist Peptide
TRVWFQPYSVQR

SEQ ID NO: 101 - protein, 12 aa, Artificial Sequence
  IL-1 Antagonist Peptide
GRIWFQPYSVQR

SEQ ID NO: 102 - protein, 12 aa, Artificial Sequence
  IL-1 Antagonist Peptide
GRVWFQPYSVQR

SEQ ID NO: 103 - protein, 12 aa, Artificial Sequence
  IL-1 Antagonist Peptide
ARTWYQPYSVQR

SEQ ID NO: 104 - protein, 12 aa, Artificial Sequence
  IL-1 Antagonist Peptide
ARVWWQPYSVQM

SEQ ID NO: 105 - protein, 12 aa, Artificial Sequence
  IL-1 Antagonist Peptide
RLMFYQPYSVQR

SEQ ID NO: 106 - protein, 12 aa, Artificial Sequence
  IL-1 Antagonist Peptide
ESMWYQPYSVQR

SEQ ID NO: 107 - protein, 12 aa, Artificial Sequence
  IL-1 Antagonist Peptide
HFGWWQPYSVHM

SEQ ID NO: 108 - protein, 12 aa, Artificial Sequence
  IL-1 Antagonist Peptide
ARFWWQPYSVQR

SEQ ID NO: 109 - protein, 12 aa, Artificial Sequence
  IL-1 Antagonist Peptide
RLVYWQPYAPIY

SEQ ID NO: 110 - protein, 12 aa, Artificial Sequence
  IL-1 Antagonist Peptide
RLVYWQPYSYQT

SEQ ID NO: 111 - protein, 12 aa, Artificial Sequence
  IL-1 Antagonist Peptide
RLVYWQPYSLPI

SEQ ID NO: 112 - protein, 12 aa, Artificial Sequence
  IL-1 Antagonist Peptide
RLVYWQPYSVQA

SEQ ID NO: 113 - protein, 12 aa, Artificial Sequence
  IL-1 Antagonist Peptide
SRVWYQPYAKGL

SEQ ID NO: 114 - protein, 12 aa, Artificial Sequence
  IL-1 Antagonist Peptide
SRVWYQPYAQGL

SEQ ID NO: 115 - protein, 12 aa, Artificial Sequence
  IL-1 Antagonist Peptide
SRVWYQPYAMPL

SEQ ID NO: 116 - protein, 12 aa, Artificial Sequence
  IL-1 Antagonist Peptide
SRVWYQPYSVQA

SEQ ID NO: 117 - protein, 12 aa, Artificial Sequence
  IL-1 Antagonist Peptide
SRVWYQPYSLGL

SEQ ID NO: 118 - protein, 12 aa, Artificial Sequence
  IL-1 Antagonist Peptide
SRVWYQPYAREL

SEQ ID NO: 119 - protein, 12 aa, Artificial Sequence
  IL-1 Antagonist Peptide
SRVWYQPYSRQP

SEQ ID NO: 120 - protein, 12 aa, Artificial Sequence
  IL-1 Antagonist Peptide
SRVWYQPYFVQP

SEQ ID NO: 121 - protein, 12 aa, Artificial Sequence
  IL-1 Antagonist Peptide
EYEWYQPYALPL

SEQ ID NO: 122 - protein, 12 aa, Artificial Sequence
  IL-1 Antagonist Peptide
IPEYWQPYALPL

SEQ ID NO: 123 - protein, 12 aa, Artificial Sequence
  IL-1 Antagonist Peptide
SRIWWQPYALPL

SEQ ID NO: 124 - protein, 12 aa, Artificial Sequence
  IL-1 Antagonist Peptide
DPLFWQPYALPL

SEQ ID NO: 125 - protein, 12 aa, Artificial Sequence
  IL-1 Antagonist Peptide
SRQWVQPYALPL

SEQ ID NO: 126 - protein, 12 aa, Artificial Sequence
  IL-1 Antagonist Peptide
IRSWWQPYALPL

SEQ ID NO: 127 - protein, 11 aa, Artificial Sequence
  IL-1 Antagonist Peptide
RGYWQPYALPL

SEQ ID NO: 128 - protein, 12 aa, Artificial Sequence
  IL-1 Antagonist Peptide
RLLWVQPYALPL

SEQ ID NO: 129 - protein, 12 aa, Artificial Sequence
  IL-1 Antagonist Peptide
EYRWFQPYALPL

SEQ ID NO: 130 - protein, 12 aa, Artificial Sequence
  IL-1 Antagonist Peptide
DAYWVQPYALPL

SEQ ID NO: 131 - protein, 12 aa, Artificial Sequence
  IL-1 Antagonist Peptide
WSGYFQPYALPL

SEQ ID NO: 132 - protein, 12 aa, Artificial Sequence
  IL-1 Antagonist Peptide
NIEFWQPYALPL

SEQ ID NO: 133 - protein, 12 aa, Artificial Sequence
  IL-1 Antagonist Peptide
TRDWVQPYALPL

SEQ ID NO: 134 - protein, 12 aa, Artificial Sequence
  IL-1 Antagonist Peptide
DSSWYQPYALPL

SEQ ID NO: 135 - protein, 12 aa, Artificial Sequence
  IL-1 Antagonist Peptide
IGNWYQPYALPL

SEQ ID NO: 136 - protein, 12 aa, Artificial Sequence
  IL-1 Antagonist Peptide
NLRWDQPYALPL

SEQ ID NO: 137 - protein, 12 aa, Artificial Sequence
  IL-1 Antagonist Peptide
LPEFWQPYALPL

SEQ ID NO: 138 - protein, 12 aa, Artificial Sequence
  IL-1 Antagonist Peptide
DSYWWQPYALPL

SEQ ID NO: 139 - protein, 12 aa, Artificial Sequence
  IL-1 Antagonist Peptide
RSQYYQPYALPL

SEQ ID NO: 140 - protein, 12 aa, Artificial Sequence
  IL-1 Antagonist Peptide
ARFWLQPYALPL

SEQ ID NO: 141 - protein, 12 aa, Artificial Sequence
  IL-1 Antagonist Peptide
NSYFWQPYALPL

SEQ ID NO: 142 - protein, 12 aa, Artificial Sequence
  IL-1 Antagonist Peptide
RFMYWQPYSVQR

SEQ ID NO: 143 - protein, 12 aa, Artificial Sequence
  IL-1 Antagonist Peptide
AHLFWQPYSVQR

SEQ ID NO: 144 - protein, 9 aa, Artificial Sequence
  IL-1 Antagonist Peptide
WWQPYALPL

SEQ ID NO: 145 - protein, 9 aa, Artificial Sequence
  IL-1 Antagonist Peptide
YYQPYALPL

SEQ ID NO: 146 - protein, 9 aa, Artificial Sequence
  IL-1 Antagonist Peptide
YFQPYALGL

SEQ ID NO: 147 - protein, 10 aa, Artificial Sequence
  IL-1 Antagonist Peptide
YWYQPYALPL

SEQ ID NO: 148 - protein, 10 aa, Artificial Sequence
  IL-1 Antagonist Peptide
RWWQPYATPL

SEQ ID NO: 149 - protein, 10 aa, Artificial Sequence
  IL-1 Antagonist Peptide
GWYQPYALGF

SEQ ID NO: 150 - protein, 10 aa, Artificial Sequence
  IL-1 Antagonist Peptide
YWYQPYALGL

SEQ ID NO: 151 - protein, 10 aa, Artificial Sequence
  IL-1 Antagonist Peptide
IWYQPYAMPL

SEQ ID NO: 152 - protein, 10 aa, Artificial Sequence
  IL-1 Antagonist Peptide
SNMQPYQRLS

SEQ ID NO: 153 - protein, 20 aa, Artificial Sequence
  IL-1 Antagonist Peptide
TFVYWQPYAVGLPAAETACN

SEQ ID NO: 154 - protein, 20 aa, Artificial Sequence
  IL-1 Antagonist Peptide
TFVYWQPYSVQMTITGKVTM

SEQ ID NO: 155 - protein, 20 aa, Artificial Sequence
  IL-1 Antagonist Peptide
  misc_feature (12)..(13): Xaa can be any naturally occurring amino acid
  misc_feature (16)..(16): Xaa can be any naturally occurring amino acid
TFVYWQPYSSHXXVPXGFPL

SEQ ID NO: 156 - protein, 20 aa, Artificial Sequence
  IL-1 Antagonist Peptide
TFVYWQPYYGNPQWAIHVRH

SEQ ID NO: 157 - protein, 20 aa, Artificial Sequence
  IL-1 Antagonist Peptide
TFVYWQPYVLLELPEGAVRA

SEQ ID NO: 158 - protein, 20 aa, Artificial Sequence
  IL-1 Antagonist Peptide
TFVYWQPYVDYVWPIPIAQV

SEQ ID NO: 159 - protein, 11 aa, Artificial Sequence
  IL-1 Antagonist Peptide
GWYQPYVDGWR

SEQ ID NO: 160 - protein, 12 aa, Artificial Sequence
  IL-1 Antagonist Peptide
RWEQPYVKDGWS

SEQ ID NO: 161 - protein, 12 aa, Artificial Sequence
  IL-1 Antagonist Peptide
EWYQPYALGWAR

SEQ ID NO: 162 - protein, 10 aa, Artificial Sequence
  IL-1 Antagonist Peptide
GWWQPYARGL

SEQ ID NO: 163 - protein, 12 aa, Artificial Sequence
  IL-1 Antagonist Peptide
LFEQPYAKALGL

SEQ ID NO: 164 - protein, 12 aa, Artificial Sequence
  IL-1 Antagonist Peptide
GWEQPYARGLAG

SEQ ID NO: 165 - protein, 12 aa, Artificial Sequence
  IL-1 Antagonist Peptide
AWVQPYATPLDE

SEQ ID NO: 166 - protein, 12 aa, Artificial Sequence
  IL-1 Antagonist Peptide
MWYQPYSSQPAE

SEQ ID NO: 167 - protein, 12 aa, Artificial Sequence
  IL-1 Antagonist Peptide
GWTQPYSQQGEV

SEQ ID NO: 168 - skipped

SEQ ID NO: 169 - protein, 11 aa, Artificial Sequence
  IL-1 Antagonist Peptide
PWIQPYARGFG

SEQ ID NO: 170 - protein, 12 aa, Artificial Sequence
  IL-1 Antagonist Peptide
RPLYWQPYSVQV

SEQ ID NO: 171 - protein, 12 aa, Artificial Sequence
  IL-1 Antagonist Peptide
TLIYWQPYSVQI

SEQ ID NO: 172 - protein, 12 aa, Artificial Sequence
  IL-1 Antagonist Peptide
RFDYWQPYSDQT

SEQ ID NO: 173 - protein, 12 aa, Artificial Sequence
  IL-1 Antagonist Peptide
WHQFVQPYALPL

SEQ ID NO: 174 - protein, 17 aa, Artificial Sequence
  IL-1 Antagonist Peptide
EWDSVYWQPYSVQTLLR

SEQ ID NO: 175 - protein, 17 aa, Artificial Sequence
  IL-1 Antagonist Peptide
WEQNVYWQPYSVQSFAD

SEQ ID NO: 176 - protein, 16 aa, Artificial Sequence
  IL-1 Antagonist Peptide
SDVVYWQPYSVQSLEM

SEQ ID NO: 177 - protein, 17 aa, Artificial Sequence
  IL-1 Antagonist Peptide
YYDGVYWQPYSVQVMPA

SEQ ID NO: 178 - protein, 12 aa, Artificial Sequence
  IL-1 Antagonist Peptide
SDIWYQPYALPL

SEQ ID NO: 179 - protein, 12 aa, Artificial Sequence
  IL-1 Antagonist Peptide
QRIWWQPYALPL

SEQ ID NO: 180 - protein, 12 aa, Artificial Sequence
  IL-1 Antagonist Peptide
SRIWWQPYALPL

SEQ ID NO: 181 - protein, 12 aa, Artificial Sequence
  IL-1 Antagonist Peptide
RSLYWQPYALPL

SEQ ID NO: 182 - protein, 12 aa, Artificial Sequence
  IL-1 Antagonist Peptide
TIIWEQPYALPL

SEQ ID NO: 183 - protein, 12 aa, Artificial Sequence
  IL-1 Antagonist Peptide
WETWYQPYALPL

SEQ ID NO: 184 - protein, 12 aa, Artificial Sequence
  IL-1 Antagonist Peptide
SYDWEQPYALPL

SEQ ID NO: 185 - protein, 12 aa, Artificial Sequence
  IL-1 Antagonist Peptide
SRIWCQPYALPL

SEQ ID NO: 186 - protein, 12 aa, Artificial Sequence
  IL-1 Antagonist Peptide
EIMFWQPYALPL

SEQ ID NO: 187 - protein, 12 aa, Artificial Sequence
  IL-1 Antagonist Peptide
DYVWQQPYALPL

SEQ ID NO: 188 - protein, 15 aa, Artificial Sequence
  IL-1 Antagonist Peptide
MDLLVQWYQPYALPL

SEQ ID NO: 189 - protein, 15 aa, Artificial Sequence
  IL-1 Antagonist Peptide
GSKVILWYQPYALPL

SEQ ID NO: 190 - protein, 15 aa, Artificial Sequence
  IL-1 Antagonist Peptide
RQGANIWYQPYALPL

SEQ ID NO: 191 - protein, 15 aa, Artificial Sequence
  IL-1 Antagonist Peptide
GGGDEPWYQPYALPL

SEQ ID NO: 192 - protein, 15 aa, Artificial Sequence
  IL-1 Antagonist Peptide
SQLERTWYQPYALPL

SEQ ID NO: 193 - protein, 15 aa, Artificial Sequence
  IL-1 Antagonist Peptide
ETWVREWYQPYALPL

SEQ ID NO: 194 - protein, 15 aa, Artificial Sequence
  IL-1 Antagonist Peptide
KKGSTQWYQPYALPL

SEQ ID NO: 195 - protein, 15 aa, Artificial Sequence
  IL-1 Antagonist Peptide
LQARMNWYQPYALPL

SEQ ID NO: 196 - protein, 15 aa, Artificial Sequence
  IL-1 Antagonist Peptide
EPRSQKWYQPYALPL

SEQ ID NO: 197 - protein, 15 aa, Artificial Sequence
  IL-1 Antagonist Peptide
VKQKWRWYQPYALPL

SEQ ID NO: 198 - protein, 15 aa, Artificial Sequence
  IL-1 Antagonist Peptide
LRRHDVWYQPYALPL

SEQ ID NO: 199 - protein, 15 aa, Artificial Sequence
  IL-1 Antagonist Peptide
RSTASIWYQPYALPL

SEQ ID NO: 200 - protein, 15 aa, Artificial Sequence
  IL-1 Antagonist Peptide
ESKEDQWYQPYALPL

SEQ ID NO: 201 - protein, 15 aa, Artificial Sequence
  IL-1 Antagonist Peptide
EGLTMKWYQPYALPL

SEQ ID NO: 202 - protein, 15 aa, Artificial Sequence
  IL-1 Antagonist Peptide
EGSREGWYQPYALPL

SEQ ID NO: 203 - protein, 12 aa, Artificial Sequence
  IL-1 Antagonist Peptide
VIEWWQPYALPL

SEQ ID NO: 204 - protein, 12 aa, Artificial Sequence
  IL-1 Antagonist Peptide
VWYWEQPYALPL

SEQ ID NO: 205 - protein, 12 aa, Artificial Sequence
  IL-1 Antagonist Peptide
ASEWWQPYALPL

SEQ ID NO: 206 - protein, 12 aa, Artificial Sequence
  IL-1 Antagonist Peptide
FYEWWQPYALPL

SEQ ID NO: 207 - protein, 12 aa, Artificial Sequence
  IL-1 Antagonist Peptide
EGWWVQPYALPL

SEQ ID NO: 208 - protein, 12 aa, Artificial Sequence
  IL-1 Antagonist Peptide
WGEWLQPYALPL

SEQ ID NO: 209 - protein, 12 aa, Artificial Sequence
  IL-1 Antagonist Peptide
DYVWEQPYALPL

SEQ ID NO: 210 - protein, 12 aa, Artificial Sequence
  IL-1 Antagonist Peptide
AHTWWQPYALPL

SEQ ID NO: 211 - protein, 12 aa, Artificial Sequence
  IL-1 Antagonist Peptide
FIEWFQPYALPL

SEQ ID NO: 212 - protein, 12 aa, Artificial Sequence
  IL-1 Antagonist Peptide
WLAWEQPYALPL

SEQ ID NO: 213 - protein, 12 aa, Artificial Sequence
  IL-1 Antagonist Peptide
VMEWWQPYALPL

SEQ ID NO: 214 - protein, 11 aa, Artificial Sequence
  IL-1 Antagonist Peptide
ERMWQPYALPL

SEQ ID NO: 215 - protein, 12 aa, Artificial Sequence
  IL-1 Antagonist Peptide
  misc_feature (2)..(3): Xaa can be any naturally occurring amino acid
  misc_feature (5)..(6): Xaa can be any naturally occurring amino acid
NXXWXXPYALPL

SEQ ID NO: 216 - protein, 12 aa, Artificial Sequence
  IL-1 Antagonist Peptide
WGNWYQPYALPL

SEQ ID NO: 217 - protein, 12 aa, Artificial Sequence
  IL-1 Antagonist Peptide
TLYWEQPYALPL

SEQ ID NO: 218 - protein, 12 aa, Artificial Sequence
  IL-1 Antagonist Peptide
VWRWEQPYALPL

SEQ ID NO: 219 - protein, 11 aa, Artificial Sequence
  IL-1 Antagonist Peptide
LLWTQPYALPL

SEQ ID NO: 220 - protein, 12 aa, Artificial Sequence
  IL-1 Antagonist Peptide
  misc_feature (5)..(6): Xaa can be any naturally occurring amino acid
SRIWXXPYALPL

SEQ ID NO: 221 - protein, 12 aa, Artificial Sequence
  IL-1 Antagonist Peptide
SDIWYQPYALPL

SEQ ID NO: 222 - protein, 12 aa, Artificial Sequence
  IL-1 Antagonist Peptide
  misc_feature (5)..(6): Xaa can be any naturally occurring amino acid
WGYYXXPYALPL

SEQ ID NO: 223 - protein, 12 aa, Artificial Sequence
  IL-1 Antagonist Peptide
TSGWYQPYALPL

SEQ ID NO: 224 - protein, 12 aa, Artificial Sequence
  IL-1 Antagonist Peptide
  misc_feature (5)..(6): Xaa can be any naturally occurring amino acid
VHPYXXPYALPL

SEQ ID NO: 225 - protein, 12 aa, Artificial Sequence
  IL-1 Antagonist Peptide
EHSYFQPYALPL

SEQ ID NO: 226 - protein, 12 aa, Artificial Sequence
  IL-1 Antagonist Peptide
  misc_feature (1)..(2): Xaa can be any naturally occurring amino acid
XXIWYQPYALPL

SEQ ID NO: 227 - protein, 12 aa, Artificial Sequence
  IL-1 Antagonist Peptide
AQLHSQPYALPL

SEQ ID NO: 228 - protein, 12 aa, Artificial Sequence
  IL-1 Antagonist Peptide
WANWFQPYALPL

SEQ ID NO: 229 - protein, 11 aa, Artificial Sequence
  IL-1 Antagonist Peptide
SRLYSQYALPL

SEQ ID NO: 230 - protein, 12 aa, Artificial Sequence
  IL-1 Antagonist Peptide
GVTFSQPYALPL

SEQ ID NO: 231 - protein, 12 aa, Artificial Sequence
  IL-1 Antagonist Peptide
SIVWSQPYALPL

SEQ ID NO: 232 - protein, 12 aa, Artificial Sequence
  IL-1 Antagonist Peptide
SRDLVQPYALPL

SEQ ID NO: 233 - protein, 17 aa, Artificial Sequence
  IL-1 Antagonist Peptide
HWGHVYWQPYSVQDDLG

SEQ ID NO: 234 - protein, 17 aa, Artificial Sequence
  IL-1 Antagonist Peptide
SWHSVYWQPYSVQSVPE

SEQ ID NO: 235 - protein, 17 aa, Artificial Sequence
  IL-1 Antagonist Peptide
WRDSVYWQPYSVQPESA

SEQ ID NO: 236 - protein, 17 aa, Artificial Sequence
  IL-1 Antagonist Peptide
TWDAVYWQPYSVQKWLD

SEQ ID NO: 237 - protein, 17 aa, Artificial Sequence
  IL-1 Antagonist Peptide
TPPWVYWQPYSVQSLDP

SEQ ID NO: 238 - protein, 17 aa, Artificial Sequence
  IL-1 Antagonist Peptide
YWSSVYWQPYSVQSVHS

SEQ ID NO: 239 - protein, 10 aa, Artificial Sequence
  IL-1 Antagonist Peptide
YWYQPYALGL

SEQ ID NO: 240 - protein, 10 aa, Artificial Sequence
  IL-1 Antagonist Peptide
YWYQPYALPL

SEQ ID NO: 241 - protein, 10 aa, Artificial Sequence
  IL-1 Antagonist Peptide
EWIQPYATGL

SEQ ID NO: 242 - protein, 10 aa, Artificial Sequence
  IL-1 Antagonist Peptide
NWEQPYAKPL

SEQ ID NO: 243 - protein, 10 aa, Artificial Sequence
  IL-1 Antagonist Peptide
AFYQPYALPL

SEQ ID NO: 244 - protein, 10 aa, Artificial Sequence
  IL-1 Antagonist Peptide
FLYQPYALPL

SEQ ID NO: 245 - protein, 10 aa, Artificial Sequence
  IL-1 Antagonist Peptide
VCKQPYLEWC

SEQ ID NO: 246 - protein, 21 aa, Artificial Sequence
  IL-1 Antagonist Peptide
ETPFTWEESNAYYWQPYALPL

SEQ ID NO: 247 - protein, 21 aa, Artificial Sequence
  IL-1 Antagonist Peptide
QGWLTWQDSVDMYWQPYALPL

SEQ ID NO: 248 - protein, 21 aa, Artificial Sequence
  IL-1 Antagonist Peptide
FSEAGYTWPENTYWQPYALPL

SEQ ID NO: 249 - protein, 21 aa, Artificial Sequence
  IL-1 Antagonist Peptide
TESPGGLDWAKIYWQPYALPL

SEQ ID NO: 250 - protein, 21 aa, Artificial Sequence
  IL-1 Antagonist Peptide
DGYDRWRQSGERYWQPYALPL

SEQ ID NO: 251 - protein, 21 aa, Artificial Sequence
  IL-1 Antagonist Peptide
TANVSSFEWTPGYWQPYALPL

SEQ ID NO: 252 - protein, 21 aa, Artificial Sequence
  IL-1 Antagonist Peptide
SVGEDHNFWTSEYWQPYALPL

SEQ ID NO: 253 - protein, 21 aa, Artificial Sequence
  IL-1 Antagonist Peptide
MNDQTSEVSTFPYWQPYALPL

SEQ ID NO: 254 - protein, 21 aa, Artificial Sequence
  IL-1 Antagonist Peptide
SWSEAFEQPRNLYWQPYALPL

SEQ ID NO: 255 - protein, 21 aa, Artificial Sequence
  IL-1 Antagonist Peptide
QYAEPSALNDWGYWQPYALPL

SEQ ID NO: 256 - protein, 21 aa, Artificial Sequence
  IL-1 Antagonist Peptide
NGDWATADWSNYYWQPYALPL

SEQ ID NO: 257 - protein, 15 aa, Artificial Sequence
  IL-1 Antagonist Peptide
THDEHIYWQPYALPL

SEQ ID NO: 258 - protein, 21 aa, Artificial Sequence
  IL-1 Antagonist Peptide
MLEKTYTTWTPGYWQPYALPL

SEQ ID NO: 259 - protein, 20 aa, Artificial Sequence
  IL-1 Antagonist Peptide
WSDPLTRDADLYWQPYALPL

SEQ ID NO: 260 - protein, 21 aa, Artificial Sequence
  IL-1 Antagonist Peptide
SDAFTTQDSQAMYWQPYALPL

SEQ ID NO: 261 - protein, 21 aa, Artificial Sequence
  IL-1 Antagonist Peptide
GDDAAWRTDSLTYWQPYALPL

SEQ ID NO: 262 - protein, 21 aa, Artificial Sequence
  IL-1 Antagonist Peptide
AIIRQLYRWSEMYWQPYALPL

SEQ ID NO: 263 - protein, 21 aa, Artificial Sequence
  IL-1 Antagonist Peptide
ENTYSPNWADSMYWQPYALPL

SEQ ID NO: 264 - protein, 21 aa, Artificial Sequence
  IL-1 Antagonist Peptide
MNDQTSEVSTFPYWQPYALPL

SEQ ID NO: 265 - protein, 21 aa, Artificial Sequence
  IL-1 Antagonist Peptide
SVGEDHNFWTSEYWQPYALPL

SEQ ID NO: 266 - protein, 20 aa, Artificial Sequence
  IL-1 Antagonist Peptide
QTPFTWEESNAYYWQPYALP

SEQ ID NO: 267 - protein, 21 aa, Artificial Sequence
  IL-1 Antagonist Peptide
ENPFTWQESNAYYWQPYALPL

SEQ ID NO: 268 - protein, 21 aa, Artificial Sequence
  IL-1 Antagonist Peptide
VTPFTWEDSNVFYWQPYALPL

SEQ ID NO: 269 - protein, 21 aa, Artificial Sequence
  IL-1 Antagonist Peptide
QIPFTWEQSNAYYWQPYALPL

SEQ ID NO: 270 - protein, 21 aa, Artificial Sequence
  IL-1 Antagonist Peptide
QAPLTWQESAAYYWQPYALPL

SEQ ID NO: 271 - protein, 21 aa, Artificial Sequence
  IL-1 Antagonist Peptide
EPTFTWEESKATYWQPYALPL

SEQ ID NO: 272 - protein, 21 aa, Artificial Sequence
  IL-1 Antagonist Peptide
TTTLTWEESNAYYWQPYALPL

SEQ ID NO: 273 - protein, 21 aa, Artificial Sequence
  IL-1 Antagonist Peptide
ESPLTWEESSALYWQPYALPL

SEQ ID NO: 274 - protein, 21 aa, Artificial Sequence
  IL-1 Antagonist Peptide
ETPLTWEESNAYYWQPYALPL

SEQ ID NO: 275 - protein, 21 aa, Artificial Sequence
  IL-1 Antagonist Peptide
EATFTWAESNAYYWQPYALPL

SEQ ID NO: 276 - protein, 21 aa, Artificial Sequence
  IL-1 Antagonist Peptide
EALFTWKESTAYYWQPYALPL

SEQ ID NO: 277 - protein, 20 aa, Artificial Sequence
  IL-1 Antagonist Peptide
STPTWEESNAYYWQPYALPL

SEQ ID NO: 278 - protein, 21 aa, Artificial Sequence
  IL-1 Antagonist Peptide
ETPFTWEESNAYYWQPYALPL

SEQ ID NO: 279 - protein, 21 aa, Artificial Sequence
  IL-1 Antagonist Peptide
KAPFTWEESQAYYWQPYALPL

SEQ ID NO: 280 - protein, 21 aa, Artificial Sequence
  IL-1 Antagonist Peptide
STSFTWEESNAYYWQPYALPL

SEQ ID NO: 281 - protein, 21 aa, Artificial Sequence
  IL-1 Antagonist Peptide
DSTFTWEESNAYYWQPYALPL

SEQ ID NO: 282 - protein, 21 aa, Artificial Sequence
  IL-1 Antagonist Peptide
YIPFTWEESNAYYWQPYALPL

SEQ ID NO: 283 - protein, 21 aa, Artificial Sequence
  IL-1 Antagonist Peptide
QTAFTWEESNAYYWQPYALPL

SEQ ID NO: 284 - protein, 21 aa, Artificial Sequence
  IL-1 Antagonist Peptide
ETLFTWEESNATYWQPYALPL

SEQ ID NO: 285 - protein, 21 aa, Artificial Sequence
  IL-1 Antagonist Peptide
VSSFTWEESNAYYWQPYALPL

SEQ ID NO: 286 - protein, 7 aa, Artificial Sequence
  IL-1 Antagonist Peptide
QPYALPL

SEQ ID NO: 287 - protein, 14 aa, Artificial Sequence
  IL-1 Antagonist Peptide
  misc_feature (9)..(9): Xaa can be any naturally occurring amino acid
PYNAPPYQXYALPL

SEQ ID NO: 288 - protein, 21 aa, Artificial Sequence
  IL-1 Antagonist Peptide
TANVSSFEWTPGYWQPYALPL

SEQ ID NO: 289 - protein, 15 aa, Artificial Sequence
  IL-1 Antagonist Peptide
FEWTPGYWQPYALPL

SEQ ID NO: 290 - protein, 15 aa, Artificial Sequence
  IL-1 Antagonist Peptide
  misc_feature (10)..(10): Xaa can be any naturally occurring amino acid
FEWTPGYWQXYALPL

SEQ ID NO: 291 - protein, 15 aa, Artificial Sequence
  IL-1 Antagonist Peptide
  misc_feature (10)..(10): Xaa can be any naturally occurring amino acid
FEWTPGYYQXYALPL

SEQ ID NO: 292 - protein, 21 aa, Artificial Sequence
  IL-1 Antagonist Peptide
ETPFTWEESNAYYWQPYALPL

SEQ ID NO: 293 - protein, 18 aa, Artificial Sequence
  IL-1 Antagonist Peptide
  misc_feature (13)..(13): Xaa can be any naturally occurring amino acid
FTWEESNAYYWQXYALPL

SEQ ID NO: 294 - protein, 16 aa, Artificial Sequence
  IL-1 Antagonist Peptide
ADVLYWQPYAPVTLWV

SEQ ID NO: 295 - protein, 17 aa, Artificial Sequence
  IL-1 Antagonist Peptide
GDVAEYWQPYALPLTSL

SEQ ID NO: 296 - protein, 18 aa, Artificial Sequence
  IL-1 Antagonist Peptide
SWTDYGYWQPYALPISGL

SEQ ID NO: 297 - protein, 15 aa, Artificial Sequence
  IL-1 Antagonist Peptide
FEWTPGYWQPYALPL

SEQ ID NO: 298 - protein, 15 aa, Artificial Sequence
  IL-1 Antagonist Peptide
  misc_feature (10)..(10): Xaa can be any naturally occurring amino acid
FEWTPGYWQXYALPL

SEQ ID NO: 299 - protein, 15 aa, Artificial Sequence
  IL-1 Antagonist Peptide
FEWTPGWYQPYALPL

SEQ ID NO: 300 - protein, 15 aa, Artificial Sequence
  IL-1 Antagonist Peptide
  misc_feature (10)..(10): Xaa can be any naturally occurring amino acid
FEWTPGWYQXYALPL

SEQ ID NO: 301 - protein, 15 aa, Artificial Sequence
  IL-1 Antagonist Peptide
FEWTPGYYQPYALPL

SEQ ID NO: 302 - protein, 15 aa, Artificial Sequence
  IL-1 Antagonist Peptide
  misc_feature (10)..(10): Xaa can be any naturally occurring amino acid
FEWTPGYYQXYALPL

SEQ ID NO: 303 - protein, 21 aa, Artificial Sequence
  IL-1 Antagonist Peptide
TANVSSFEWTPGYWQPYALPL

SEQ ID NO: 304 - protein, 18 aa, Artificial Sequence
  IL-1 Antagonist Peptide
SWTDYGYWQPYALPISGL

SEQ ID NO: 305 - protein, 21 aa, Artificial Sequence
  IL-1 Antagonist Peptide
ETPFTWEESNAYYWQPYALPL

SEQ ID NO: 306 - protein, 21 aa, Artificial Sequence
  IL-1 Antagonist Peptide
ENTYSPNWADSMYWQPYALPL

SEQ ID NO: 307 - protein, 21 aa, Artificial Sequence
  IL-1 Antagonist Peptide
SVGEDHNFWTSEYWQPYALPL

SEQ ID NO: 308 - protein, 21 aa, Artificial Sequence
  IL-1 Antagonist Peptide
DGYDRWRQSGERYWQPYALPL

SEQ ID NO: 309 - protein, 15 aa, Artificial Sequence
  IL-1 Antagonist Peptide
FEWTPGYWQPYALPL

SEQ ID NO: 310 - protein, 11 aa, Artificial Sequence
  IL-1 Antagonist Peptide
FEWTPGYWQPY

SEQ ID NO: 311 - protein, 11 aa, Artificial Sequence
  IL-1 Antagonist Peptide
  misc_feature (10)..(10): Xaa can be any naturally occurring amino acid
FEWTPGYWQXY

SEQ ID NO: 312 - protein, 10 aa, Artificial Sequence
  IL-1 Antagonist Peptide
EWTPGYWQPY

SEQ ID NO: 313 - protein, 11 aa, Artificial Sequence
  IL-1 Antagonist Peptide
  misc_feature (10)..(10): Xaa can be any naturally occurring amino acid
FEWTPGWYQXY

SEQ ID NO: 314 - protein, 11 aa, Artificial Sequence
  IL-1 Antagonist Peptide
  misc_feature (10)..(10): Xaa can be any naturally occurring amino acid
AEWTPGYWQXY

SEQ ID NO: 315 - protein, 11 aa, Artificial Sequence
  IL-1 Antagonist Peptide
  misc_feature (10)..(10): Xaa can be any naturally occurring amino acid
FAWTPGYWQXY

SEQ ID NO: 316 - protein, 11 aa, Artificial Sequence
  IL-1 Antagonist Peptide
  misc_feature (10)..(10): Xaa can be any naturally occurring amino acid
FEATPGYWQXY

SEQ ID NO: 317 - protein, 11 aa, Artificial Sequence
  IL-1 Antagonist Peptide
  misc_feature (10)..(10): Xaa can be any naturally occurring amino acid
FEWAPGYWQXY

SEQ ID NO: 318 - protein, 11 aa, Artificial Sequence
  IL-1 Antagonist Peptide
  misc_feature (10)..(10): Xaa can be any naturally occurring amino acid
FEWTAGYWQXY

SEQ ID NO: 319 - protein, 11 aa, Artificial Sequence
  IL-1 Antagonist Peptide
  misc_feature (10)..(10): Xaa can be any naturally occurring amino acid
FEWTPAYWQXY

SEQ ID NO: 320 - protein, 11 aa, Artificial Sequence
  IL-1 Antagonist Peptide
  misc_feature (10)..(10): Xaa can be any naturally occurring amino acid
FEWTPGAWQXY

SEQ ID NO: 321 - protein, 11 aa, Artificial Sequence
  IL-1 Antagonist Peptide
  misc_feature (10)..(10): Xaa can be any naturally occurring amino acid
FEWTPGYAQXY

SEQ ID NO: 322 - protein, 11 aa, Artificial Sequence
  IL-1 Antagonist Peptide
  misc_feature (10)..(10): Xaa can be any naturally occurring amino acid
FEWTPGYWQXA

SEQ ID NO: 323 - protein, 11 aa, Artificial Sequence
  IL-1 Antagonist Peptide
  misc_feature (10)..(10): Xaa can be any naturally occurring amino acid
FEWTGGYWQXY

SEQ ID NO: 324 - protein, 11 aa, Artificial Sequence
  IL-1 Antagonist Peptide
  misc_feature (10)..(10): Xaa can be any naturally occurring amino acid
FEWTPGYWQXY

SEQ ID NO: 325 - protein, 11 aa, Artificial Sequence
  IL-1 Antagonist Peptide
  misc_feature (5)..(5): Xaa can be any naturally occurring amino acid
  misc_feature (10)..(10): Xaa can be any naturally occurring amino acid
FEWTXGYWQXY

SEQ ID NO: 326 - protein, 13 aa, Artificial Sequence
  IL-1 Antagonist Peptide
  misc_feature (12)..(12): Xaa can be any naturally occurring amino acid
FEWTPECGYWQXY

SEQ ID NO: 327 - protein, 13 aa, Artificial Sequence
  IL-1 Antagonist Peptide
  misc_feature (12)..(12): Xaa can be any naturally occurring amino acid
FEWTPAIBYWQXY

SEQ ID NO: 328 - protein, 13 aa, Artificial Sequence
  IL-1 Antagonist Peptide
  misc_feature (12)..(12): Xaa can be any naturally occurring amino acid
FEWTPSARWYQXY

SEQ ID NO: 329 - protein, 13 aa, Artificial Sequence
  IL-1 Antagonist Peptide
  misc_feature (12)..(12): Xaa can be any naturally occurring amino acid
FEWTSARGYWQXY

SEQ ID NO: 330 - protein, 11 aa, Artificial Sequence
  IL-1 Antagonist Peptide
  misc_feature (10)..(10): Xaa can be any naturally occurring amino acid
FEWTPNYWQXY

SEQ ID NO: 331 - protein, 11 aa, Artificial Sequence
  IL-1 Antagonist Peptide
  misc_feature (10)..(10): Xaa can be any naturally occurring amino acid
FEWTPVYWQXY

SEQ ID NO: 332 - protein, 11 aa, Artificial Sequence
  IL-1 Antagonist Peptide
  misc_feature (10)..(10): Xaa can be any naturally occurring amino acid
FEWTVPYWQXY

SEQ ID NO: 333 - protein, 13 aa, Artificial Sequence
  IL-1 Antagonist Peptide
  misc_feature (12)..(12): Xaa can be any naturally occurring amino acid
ACFEWTPGWYQXY

SEQ ID NO: 334 - protein, 13 aa, Artificial Sequence
  IL-1 Antagonist Peptide
  misc_feature (12)..(12): Xaa can be any naturally occurring amino acid
ACFEWTPGYWQXY

SEQ ID NO: 335 - protein, 14 aa, Artificial Sequence
  IL-1 Antagonist Peptide
  misc_feature (13)..(13): Xaa can be any naturally occurring amino acid
LNAPEWTPGYYQXY

SEQ ID NO: 336 - protein, 11 aa, Artificial Sequence
  IL-1 Antagonist Peptide
  misc_feature (10)..(10): Xaa can be any naturally occurring amino acid
YEWTPGYYQXY

SEQ ID NO: 337 - protein, 11 aa, Artificial Sequence
  IL-1 Antagonist Peptide
  misc_feature (10)..(10): Xaa can be any naturally occurring amino acid
FEWVPGYYQXY

SEQ ID NO: 338 - protein, 11 aa, Artificial Sequence
  IL-1 Antagonist Peptide
  misc_feature (10)..(10): Xaa can be any naturally occurring amino acid
FEWTPGYYQXY

SEQ ID NO: 339 - protein, 11 aa, Artificial Sequence
  IL-1 Antagonist Peptide
  misc_feature (10)..(10): Xaa can be any naturally occurring amino acid
FEWTPSYYQXY

SEQ ID NO: 340 - protein, 11 aa, Artificial Sequence
  IL-1 Antagonist Peptide
  misc_feature (10)..(10): Xaa can be any naturally occurring amino acid
FEWTPNYYQXY

SEQ ID NO: 341 - protein, 14 aa, Artificial Sequence
  IL-1 Antagonist Peptide
SHLYNAPQPYSVQM

SEQ ID NO: 342 - protein, 14 aa, Artificial Sequence
  IL-1 Antagonist Peptide
TLVYNAPQPYSLQT

SEQ ID NO: 343 - protein, 14 aa, Artificial Sequence
  IL-1 Antagonist Peptide
RGDYNAPQPYSVQS

SEQ ID NO: 344 - protein, 14 aa, Artificial Sequence
  IL-1 Antagonist Peptide
NMVYNAPQPYSIQT

SEQ ID NO: 345 - protein, 9 aa, Artificial Sequence
  IL-1 Antagonist Peptide
VYWQPYSVQ

SEQ ID NO: 346 - protein, 11 aa, Artificial Sequence
  IL-1 Antagonist Peptide
VYNAPQPYSVQ

SEQ ID NO: 347 - protein, 12 aa, Artificial Sequence
  IL-1 Antagonist Peptide
  misc_feature (7)..(7): Xaa can be any naturally occurring amino acid
TFVYWQXYALPL

SEQ ID NO: 348 - protein, 13 aa, Artificial Sequence
  IL-1 Antagonist Peptide
  misc_feature (10)..(10): Xaa can be any naturally occurring amino acid
FEWTPGYYQXBPA

SEQ ID NO: 349 - protein, 16 aa, Artificial Sequence
  IL-1 Antagonist Peptide
  misc_feature (1)..(1): Xaa can be any naturally occurring amino acid
  misc_feature (13)..(13): Xaa can be any naturally occurring amino acid
XAAFEWTPGYYQXBPA

SEQ ID NO: 350 - protein, 13 aa, Artificial Sequence
  IL-1 Antagonist Peptide
  misc_feature (12)..(12): Xaa can be any naturally occurring amino acid
FEWTPGYBPAQXY

SEQ ID NO: 351 - protein, 15 aa, Artificial Sequence
  IL-1 Antagonist Peptide
  misc_feature (14)..(14): Xaa can be any naturally occurring amino acid
ACFEWTPGYBPAQXY

SEQ ID NO: 352 - protein, 13 aa, Artificial Sequence
  IL-1 Antagonist Peptide
  misc_feature (12)..(12): Xaa can be any naturally occurring amino acid
FEWTPGBPAYQXY

SEQ ID NO: 353 - protein, 15 aa, Artificial Sequence
  IL-1 Antagonist Peptide
  misc_feature (14)..(14): Xaa can be any naturally occurring amino acid
ACFEWTPGBPAYQXY

SEQ ID NO: 354 - protein, 15 aa, Artificial Sequence
  IL-1 Antagonist Peptide
  misc_feature (14)..(14): Xaa can be any naturally occurring amino acid
ACFEBPATPGYYQXY

SEQ ID NO: 355 - protein, 15 aa, Artificial Sequence
  IL-1 Antagonist Peptide
  misc_feature (14)..(14): Xaa can be any naturally occurring amino acid
ACFEBPATPGYYQXY

SEQ ID NO: 356 - protein, 13 aa, Artificial Sequence
  IL-1 Antagonist Peptide
  misc_feature (12)..(12): Xaa can be any naturally occurring amino acid
BPAEWTPGYYQXY

SEQ ID NO: 357 - protein, 15 aa, Artificial Sequence
  IL-1 Antagonist Peptide
  misc_feature (14)..(14): Xaa can be any naturally occurring amino acid
ACBPAEWTPGYYQXY

SEQ ID NO: 358 - protein, 9 aa, Artificial Sequence
  IL-1 Antagonist Peptide
VYWQPYSVQ

SEQ ID NO: 359 - protein, 12 aa, Artificial Sequence
  IL-1 Antagonist Peptide
RLVYWQPYSVQR

SEQ ID NO: 360 - protein, 14 aa, Artificial Sequence
  IL-1 Antagonist Peptide
RLVYNAPQPYSVQR

SEQ ID NO: 361 - protein, 12 aa, Artificial Sequence
  IL-1 Antagonist Peptide
RLDYWQPYSVQR

SEQ ID NO: 362 - protein, 12 aa, Artificial Sequence
  IL-1 Antagonist Peptide
RLVWFQPYSVQR

SEQ ID NO: 363 - protein, 12 aa, Artificial Sequence
  IL-1 Antagonist Peptide
RLVYWQPYSIQR

SEQ ID NO: 364 - protein, 11 aa, Artificial Sequence
  IL-1 Antagonist Peptide
DNSSWYDSFLL

SEQ ID NO: 365 - protein, 11 aa, Artificial Sequence
  IL-1 Antagonist Peptide
DNTAWYESFLA

SEQ ID NO: 366 - protein, 11 aa, Artificial Sequence
  IL-1 Antagonist Peptide
DNTAWYENFLL

SEQ ID NO: 367 - protein, 17 aa, Artificial Sequence
  IL-1 Antagonist Peptide
PAREDNTAWYDSFLIWC

SEQ ID NO: 368 - protein, 17 aa, Artificial Sequence
  IL-1 Antagonist Peptide
TSEYDNTTWYEKFLASQ

SEQ ID NO: 369 - protein, 17 aa, Artificial Sequence
  IL-1 Antagonist Peptide
SQIPDNTAWYQSFLLHG

SEQ ID NO: 370 - protein, 17 aa, Artificial Sequence
  IL-1 Antagonist Peptide
SPFIDNTAWYENFLLTY

SEQ ID NO: 371 - protein, 17 aa, Artificial Sequence
  IL-1 Antagonist Peptide
EQIYDNTAWYDHFLLSY

SEQ ID NO: 372 - protein, 17 aa, Artificial Sequence
  IL-1 Antagonist Peptide
TPFIDNTAWYENFLLTY

SEQ ID NO: 373 - protein, 17 aa, Artificial Sequence
  IL-1 Antagonist Peptide
TYTYDNTAWYERFLMSY

SEQ ID NO: 374 - protein, 17 aa, Artificial Sequence
  IL-1 Antagonist Peptide
TMTQDNTAWYENFLLSY

SEQ ID NO: 375 - protein, 17 aa, Artificial Sequence
  IL-1 Antagonist Peptide
TIDNTAWYANLVQTYPQ

SEQ ID NO: 376 - protein, 17 aa, Artificial Sequence
  IL-1 Antagonist Peptide
TIDNTAWYERFLAQYPD

SEQ ID NO: 377 - protein, 17 aa, Artificial Sequence
  IL-1 Antagonist Peptide
HIDNTAWYENFLLTYTP

SEQ ID NO: 378 - protein, 17 aa, Artificial Sequence
  IL-1 Antagonist Peptide
SQDNTAWYENFLLSYKA

SEQ ID NO: 379 - protein, 17 aa, Artificial Sequence
  IL-1 Antagonist Peptide
QIDNTAWYERFLLQYNA

SEQ ID NO: 380 - protein, 17 aa, Artificial Sequence
  IL-1 Antagonist Peptide
NQDNTAWYESFLLQYNT

SEQ ID NO: 381 - protein, 17 aa, Artificial Sequence
  IL-1 Antagonist Peptide
TIDNTAWYENFLLNHNL

SEQ ID NO: 382 - protein, 17 aa, Artificial Sequence
  IL-1 Antagonist Peptide
HYDNTAWYERFLQQGWH

SEQ ID NO: 383 - protein, 21 aa, Artificial Sequence
  IL-1 Antagonist Peptide
ETPFTWEESNAYYWQPYALPL

SEQ ID NO: 384 - protein, 21 aa, Artificial Sequence
  IL-1 Antagonist Peptide
YIPFTWEESNAYYWQPYALPL

SEQ ID NO: 385 - protein, 21 aa, Artificial Sequence
  IL-1 Antagonist Peptide
DGYDRWRQSGERYWQPYALPL

SEQ ID NO: 386 - protein, 15 aa, Artificial Sequence
  IL-1 Antagonist Peptide
  misc_feature (10)..(10): Xaa can be any naturally occurring amino acid
PYLNAPPYQXYALPL

SEQ ID NO: 387 - protein, 21 aa, Artificial Sequence
  IL-1 Antagonist Peptide
TANVSSFEWTPGYWQPYALPL

SEQ ID NO: 388 - protein, 15 aa, Artificial Sequence
  IL-1 Antagonist Peptide
  misc_feature (10)..(10): Xaa can be any naturally occurring amino acid
FEWTPGYWQXYALPL

SEQ ID NO: 389 - protein, 17 aa, Artificial Sequence
  IL-1 Antagonist Peptide
FEWTPGYWQPYALPLSD

SEQ ID NO: 390 - protein, 15 aa, Artificial Sequence
  IL-1 Antagonist Peptide
  misc_feature (10)..(10): Xaa can be any naturally occurring amino acid
FEWTPGYYQXYALPL

SEQ ID NO: 391 - protein, 11 aa, Artificial Sequence
  IL-1 Antagonist Peptide
  misc_feature (10)..(10): Xaa can be any naturally occurring amino acid
FEWTPGYWQXY

SEQ ID NO: 392 - protein, 13 aa, Artificial Sequence
  IL-1 Antagonist Peptide
  misc_feature (12)..(12): Xaa can be any naturally occurring amino acid
ACFEWTPGYWQXY

SEQ ID NO: 393 - protein, 13 aa, Artificial Sequence
  IL-1 Antagonist Peptide
  misc_feature (12)..(12): Xaa can be any naturally occurring amino acid
ACFEWTPGWYQXY

SEQ ID NO: 394 - protein, 13 aa, Artificial Sequence
  IL-1 Antagonist Peptide
  misc_feature (12)..(12): Xaa can be any naturally occurring amino acid
ACFEWTPGYYQXY

SEQ ID NO: 395 - protein, 13 aa, Artificial Sequence
  IL-1 Antagonist Peptide
  misc_feature (12)..(12): Xaa can be any naturally occurring amino acid
ACFEWTPAYWQXY

SEQ ID NO: 396 - protein, 13 aa, Artificial Sequence
  IL-1 Antagonist Peptide
  misc_feature (12)..(12): Xaa can be any naturally occurring amino acid
ACFEWTPAWYQXY

SEQ ID NO: 397 - protein, 13 aa, Artificial Sequence
  IL-1 Antagonist Peptide
  misc_feature (12)..(12): Xaa can be any naturally occurring amino acid
ACFEWTPAYYQXY

SEQ ID NO: 398 - protein, 15 aa, Artificial Sequence
  IL-1 Antagonist Peptide
  misc_feature (10)..(10): Xaa can be any naturally occurring amino acid
FEWTPGYYQXYALPL

SEQ ID NO: 399 - protein, 15 aa, Artificial Sequence
  IL-1 Antagonist Peptide
  misc_feature (10)..(10): Xaa can be any naturally occurring amino acid
FEWTPGYWQXYALPL

SEQ ID NO: 400 - protein, 15 aa, Artificial Sequence
  IL-1 Antagonist Peptide
  misc_feature (10)..(10): Xaa can be any naturally occurring amino acid
FEWTPGWYQXYALPL

SEQ ID NO: 401 - protein, 21 aa, Artificial Sequence
  IL-1 Antagonist Peptide
TANVSSFEWTPGYWQPYALPL

SEQ ID NO: 402 - protein, 21 aa, Artificial Sequence
  IL-1 Antagonist Peptide
TANVSSFEWTPGYWQPYALPL

SEQ ID NO: 403 - protein, 13 aa, Artificial Sequence
  IL-1 Antagonist Peptide
  misc_feature (12)..(12): Xaa can be any naturally occurring amino acid
ACFEWTPGWYQXY

SEQ ID NO: 404 - protein, 13 aa, Artificial Sequence
  IL-1 Antagonist Peptide
  misc_feature (12)..(12): Xaa can be any naturally occurring amino acid
ACFEWTPGYYQXY

SEQ ID NO: 405 - protein, 13 aa, Artificial Sequence
  IL-1 Antagonist Peptide
  misc_feature (12)..(12): Xaa can be any naturally occurring amino acid
ACFEWTPAYWQXY

SEQ ID NO: 406 - protein, 13 aa, Artificial Sequence
  IL-1 Antagonist Peptide
  misc_feature (12)..(12): Xaa can be any naturally occurring amino acid
ACFEWTPAWYQXY

SEQ ID NO: 407 - protein, 13 aa, Artificial Sequence
  IL-1 Antagonist Peptide
  misc_feature (12)..(12): Xaa can be any naturally occurring amino acid
ACFEWTPAYYQXY

SEQ ID NO: 408 - protein, 139 aa, Homo sapiens
MEFGLSWVFLVALLRGVQCQVQLVESGGGVVQPGRSLRLSCAASGFTFSNYGMHWVRQAPGKGLEWVAGIWNDGINKYHAHSVRGRFTISRDNSKNTLYLQMNSPRAEDTAVYYCARARSFDWLLFEFWGQGTLVTVSS

SEQ ID NO: 409 - protein, 139 aa, Homo sapiens
MEFGLSWVFLVALLRGVQCQVQLVESGGGVVQPGRSLRLSCAVSGFTFSNYGMHWVRQAPGKGLEWVAAIWNDGENKHHAGSVRGRFTISRDNSKNTLYLQMNSLRAEDTAVYYCARGRYFDWLLFEYWGQGTLVTVSS

SEQ ID NO: 410 - protein, 137 aa, Homo sapiens
MGSTAILALLLAVLQGVCAEVQLMQSGAEVKKPGESLKISCKGSGYSFSFHWIAWVRQMPGKGLEWMGIIHPGASDTRYSPSFQGQVTISADNSNSATYLQWSSLKASDTAMYFCARQRELDYFDYWGQGTLVTVSS

SEQ ID NO: 411 - protein, 128 aa, Homo sapiens
MEAPAQLLFLLLLWLPDTTGEIVLTQSPATLSLSPGERATLSCRASQSVSSYLAWYQQKPGQAPRLLIYDASNRATGIPARFSGSGSGTDFTLTISSLEPEDFAVYYCQQRSNWPPLTFGGGTKVEIK

SEQ ID NO: 412 - protein, 126 aa, Homo sapiens
MSPSQLIGFLLLWVPASRGEIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPLTFGGGTKVEIK